SEQUENCE LISTING (8 sequences)

SEQ ID NO: 1 - DNA, 269 bp, SARS-CoV-2
  source 1..269: mol_type = unassigned DNA; note = ORF1 fragment
ccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaaga

SEQ ID NO: 2 - DNA, 237 bp, SARS-CoV-2
  source 1..237: mol_type = unassigned DNA; note = Gene N fragment
atgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctac

SEQ ID NO: 3 - DNA, 48 bp, synthetic construct
  misc_feature 1..48: note = Forward Inner Primer (FIP)
  source 1..48: mol_type = other DNA
aggtgagggttttctacatcactatattggaacaagcaaattctatgg

SEQ ID NO: 4 - DNA, 43 bp, synthetic construct
  misc_feature 1..43: note = Backward Inner Primer (BIP)
  source 1..43: mol_type = other DNA
atgggttgggattatcctaaatgtgtgcgagcaagaacaagtg

SEQ ID NO: 5 - DNA, 20 bp, synthetic construct
  misc_feature 1..20: note = Forward Outer Primer (F3
  source 1..20: mol_type = other DNA
ccactagaggagctactgta

SEQ ID NO: 6 - DNA, 18 bp, synthetic construct
  misc_feature 1..18: note = Backward Outer Primer (B3)
  source 1..18: mol_type = other DNA
tgacaagctacaacacgt

SEQ ID NO: 7 - DNA, 25 bp, synthetic construct
  misc_feature 1..25: note = Forward Loop Primer (FL)
  source 1..25: mol_type = other DNA
cagtttttaacatgttgtgccaacc

SEQ ID NO: 8 - DNA, 21 bp, synthetic construct
  misc_feature 1..21: note = Backward Loop Primer (BL)
  source 1..21: mol_type = other DNA
tagagccatgcctaacatgct

(19) United States
(12) Patent Application Publication
(10) Pub. No.: US 2023/0074766 A1
(43) Pub. Date: Mar. 9, 2023

(54) Method of Measuring the pH of a Sample

(71) Applicant: Diagonal Bio AB, Lund (SE)

(72) Inventors: Kushagr Punyani, Lund (SE); Per Andreas Nyberg, Lund (SE); Sindre Sopstad, Horten (NO); Martin Peacock, Royston (GB); Linhongjia Xiong, Royston (GB); Jae Yen Shin, Lund (SE)

(21) Appl. No.: 17/949,554

(22) Filed: Sep. 21, 2022

Related U.S. Application Data
(63) Continuation of application No. PCT/EP2021/058946, filed on Apr. 6, 2021.

(30) Foreign Application Priority Data
Apr. 3, 2020 (EP) 20168019.6
Sep. 22, 2020 (EP) 20197572.9

(51) Int. Cl. C12Q 1/6844; G01N 21/80; C12Q 1/6825; G01N 27/416; G01N 27/48

(52) U.S. Cl. CPC C12Q 1/6844; G01N 21/80; C12Q 1/6825; G01N 27/4167; G01N 27/48

(57) ABSTRACT

Disclosed herein is a more sensitive and accurate method of monitoring the pH of a solution, wherein the pH of the solution is quantified as a function of the electrochemical response of the solution in a two or three-electrode electrochemical cell, wherein the solution comprises a compound capable of undergoing a change in its oxidation state and/or structural conformation as a function of the pH of the solution. Also disclosed are highly accelerated methods and processes enabling analysis of specific polynucleotide sequences in a sample, e.g. a biological sample. The methods disclosed herein are, for example, useful for rapid screening of a large amount of samples in a point-of-care setting.

### CROSS-REFERENCE TO RELATED APPLICATIONS

This application is a continuation of PCT/EP2021/058946, filed Apr. 6, 2021, which claims priority to EP 20168019.6 filed Apr. 3, 2020, and EP 20197572.9 filed Sep. 20, 2020, all of which are herein incorporated by reference in their entirety.

### REFERENCE TO A SEQUENCE LISTING SUBMITTED VIA PATENTCENTER

The content of the XML file of the sequence listing named “20220921_312238_002US1_ST26” which is 11,298 bytes in size was created on Sep. 21, 2022 and electronically submitted via PatentCenter herewith the application is incorporated herein by reference in its entirety.

### TECHNICAL FIELD

The present invention provides a more sensitive and accurate method of monitoring the pH of a solution, wherein the pH of the solution is quantified as a function of the electrochemical response of the solution in a two or three-electrode electrochemical cell, wherein the solution comprises a compound capable of undergoing a change in its oxidation state and/or structural conformation as a function of the pH of the solution.

The present invention also provides highly accelerated methods and processes enabling analysis of specific polynucleotide sequences in a sample, e.g. a biological sample. The methods of the inventions are, for example, useful for rapid screening of a large amount of samples in a point-of-care setting.

### BACKGROUND

Nucleic acid amplification methods are crucial for medical and environmental diagnostics and often considered to be the gold standards in several diagnostic applications. Traditional methods such as Polymerase Chain Reaction, Quantitative Polymerase Chain Reaction, Reverse Transcriptase Polymerase Chain Reaction and Quantitative Reverse Transcriptase Polymerase Chain Reaction are largely dependent on thermal cycling for actuation of amplification due to the need of different temperatures for denaturation, annealing and extension by commonly used polymerases. The technological issues and the resultant high implementation cost of these methods has hampered their point-of-care use. Isothermal amplification methods such as Branched Rolling Circle Amplification and Loop Mediated Isothermal Amplification are alternatives.

Quantification of the nucleic acid amplicons and real time monitoring of amplification is indirect and requires labels, which are non-essential for the amplification reaction. These may include fluorescent dyes or chromophores attached to oligonucleotides or primers, DNA-intercalating dyes, groove binders, dyes binding to nucleotides, phosphate groups, ribose or deoxyribose groups and the nitrogen bases endogenous to the nucleic acids. These dyes or complexes may exhibit a spectral shift upon binding to the DNA, undergo fluorescence resonance energy transfer, and/or a change in magnitude of fluorescence when excited by light of suitable wavelengths, thereby facilitating optical detection. This method of quantification needs expensive and sensitive optics, thereby further limiting the use of molecular diagnostics for point-of-care applications.

Several of these DNA binding molecules, such as Hoechst 33258, Methylene Blue, nucleic acid-binding transition metal complexes such as ruthenium, osmium or platinum containing complexes are also electroactive and may facilitate indirect electrochemical detection by continuous sequestration of said molecules to the newly generated amplicons upon successful amplification, and the consequent reduced conductivity of the reaction mixture.

Alternative electrochemical detection methodologies involve immobilization of the enzymes, primers or probe oligonucleotides on the electrode surface thereby actuating quantification of the amplicons or byproducts. Most of these methods are either indirect or require expensive devices.

Recent development in Loop mediated isothermal amplification technology include colorimetric detection of RNA and DNA targets in a weakly buffered reaction mixture, mediated by a pH sensitive dye (WO 2017/209920, WO 2014/031783). The weak buffering due to low concentration of Tris is overcome by the protons released during amplification and results in an end-point detection indicated by the color change of the pH indicator. The method has been deployed for point-of-care testing, but suffers from long reaction times, low sensitivity for low nucleic acid concentrations, and the need of expensive optical devices for quantification.

### SUMMARY

The present invention provides a more sensitive and accurate method of monitoring the pH of a solution, wherein the pH of the solution is quantified as a function of the electrochemical response of the solution in a three-electrode electrochemical cell, wherein the solution comprises a quinone, a quinone derivative, and/or a pH indicator. This more sensitive and accurate method has been found to provide rapid and accurate results in determining whether or not a target polynucleotide is present in a sample and also quantifying the target polynucleotide present in the sample.

The present invention, therefore, also provides a method of quantifying a target polynucleotide in a sample. The method can be performed with high sensitivity without the need of optical devices for quantification and with a low reaction time. In particular, the quantification of the target polynucleotide can be derived from the rate of change of the electrochemical response (e.g., current and/or potential and/or impedance) of the electrochemical cell, such as the three-electrode electrochemical cell.

Thus, the present invention provides highly accelerated methods and processes enabling analysis of specific polynucleotide sequences in a sample. The methods are indeed useful for rapid screening of a large amount of samples in a point-of-care setting.

This is made possible because of inventive exploration of nucleic acid amplification techniques (such as LAMP) combined with electrochemical quantification of a signaling substance using an electrochemical cell, such as a three-electrode electrochemical cell or an electrochemical cell having multiple film electrodes.

The invention is defined in the claims attached hereto.

According to the first aspect of the invention there is provided a method of measuring the pH of a solution, wherein the method comprises the steps of:
providing a solution comprising a quinone, a quinone derivative, and/or a pH indicator;
applying the solution to a three-electrode electrochemical cell;
measuring an electrochemical response of the electrochemical cell; and
quantifying the pH of the solution as a function of the electrochemical response of the electrochemical cell.

According to another aspect of the invention there is provided a use of a three-electrode electrochemical cell for measuring the pH of a solution, said use comprising:
providing a solution comprising a quinone, a quinone derivative, a pH indicator, or combinations thereof;
applying the solution to the three-electrode electrochemical cell;
measuring an electrochemical response of the electrochemical cell; and
quantifying the pH of the solution as a function of the electrochemical response of the electrochemical cell.

According to another aspect of the invention there is provided a use of a three-electrode electrochemical cell for monitoring a nucleic acid amplification reaction, said use comprising:
providing a solution comprising a quinone, a quinone derivative, a pH indicator, or combinations thereof;
providing a LAMP mixture comprising: a sample comprising a target polynucleotide, at least two, such as at least four primers each flanking the target polynucleotide and LAMP reagents;
applying the solution and the LAMP mixture to the three-electrode electrochemical cell;
measuring an electrochemical response of the electrochemical cell; and
quantifying the pH of the solution as a function of the electrochemical response of the electrochemical cell.

According to another aspect of the invention there is provided a system for measuring the pH of a solution, the system comprising:
a. An instrument comprising a potentiostat; and
   i. A first receptacle comprising a three-electrode electrochemical cell, and a second receptacle comprising a quinone, a quinone derivative, and/or a pH indicator, or
   ii. A first receptacle comprising a three-electrode electrochemical cell and a quinone, a quinone derivative, and/or a pH indicator,
   wherein the potentiostat is configured to measure an electrochemical response of the electrochemical cell.

### DESCRIPTION OF DRAWINGS

FIG. 1: A schematic drawing of LAMP primers and the polynucleotide sequences they anneal to. Arrows indicate where a primer anneals and inverse shades of grey indicates complementarity. FIP: forward inner primer. BIP: backward inner primer. F3: forward outer primer. B3: backward outer primer. FL: forward loop primer. BL: backward loop primer.

FIG. 2: (A) A fluid or fluidized or resuspended biological sample, in a suitable carrier or transport medium (if any) after suitable processing (if any) is mixed with (B) LAMP primers and (C) WarmStart Colorimetric LAMP 2× Master Mix from New England Biolabs Inc, and immediately added on to (D) a pH sensing screen printed electrode or a screen printed electrode which is at 59-75° C. and connected to a potentiostat and voltammetric or other electrochemical measurements are run for 1-90 minutes. The (E) change in signal over time is measured. A zero change in signal in 1-90 minutes compared to a parallelized or pre-calibrated non-template control indicates (F) the absence of the template nucleic acid. A significant non-zero change in signal indicates (G) the presence of the template nucleic acid, and the rate of change of this value is used to quantify the amount of the template nucleic acid in the biological sample using a pre-determined calibration curve specific to the LAMP primers and the biological sample and its processing.

FIG. 3: A cyclic voltammogram of phenol red. Three peaks are identified at −0.35V, 0.2V and 0.55V respectively.

FIG. 4: Cathodic cyclic voltammetry scans of solutions containing 1.0 mM phenol red buffered at pH 7.9 in 0.1 M PBS on a bare glassy carbon electrode at scan rate of 100, 300, 500, 700, and 900 mV/s, initiated from 1.5 V vs. Ag/AgCl. Inset plot shows the current dependence upon scan rate and square root of scan rate, respectively.

FIG. 5: Cathodic cyclic voltammetry scans of solutions containing 1.0 mM phenol red (buffered at pH 7.9 in 0.1 M PBS) on a bare glassy carbon electrode over different switching potentials, at a scan rate of 100 mV/s initiated from 1.5 V vs. Ag/AgCl. Inset plot reveals the ratio of ipc/ipa near −0.75 V varied according to the switching potential.

FIG. 6: Anodic cyclic voltammetry scans of solutions containing 1.0 mM phenol red buffered at pH (a) 3.9, (b) 7.9, and (c) 11.9 with 0.1 M PBS on a bare glassy carbon electrode, respectively; (d) represents the scan of the solution (b) initiated from more positive potential. The cathodic cyclic voltammetry scans of those corresponding pH solutions are noted as a′, b′, and c′.

FIG. 7a-d: Example of squarewave voltammetry of phenol red in the pH range of 6.5 to 8.5, and at different concentrations of Phenol Red. FIGS. 7b and 7d show peak positions (voltage) and heights (current) versus concentrations of phenol red.

FIG. 8: An example of in-house algorithm for correlating the pH with phenol red output signal, where an optimised algorithm is used to nest both the peak potential (peak identified at around 0.5V (vs Ag/AgCl) and peak current.

FIG. 9: Voltammogram showing the peaks observed in cyclic voltammetry of phenol red solution.

FIG. 10: Schematic showing the design of sensor environment controller.

### DETAILED DESCRIPTION

### Definitions

The term “nucleotide sense strand” as used herein refers to a DNA or RNA strand that is complementary to a nucleotide antisense strand.

The term “nucleotide antisense strand” as used herein refers to a DNA or RNA strand that is complementary to a nucleotide sense strand.

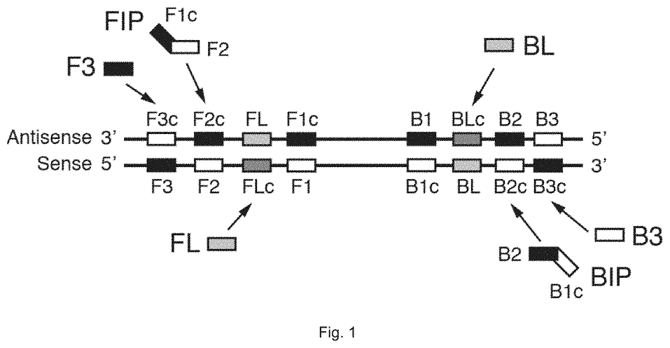

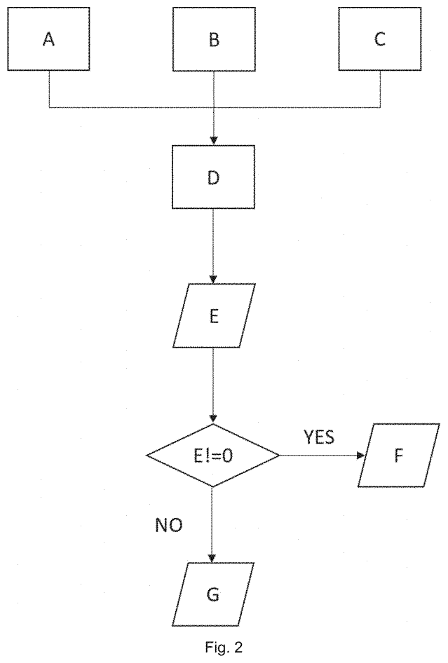

The term “forward inner primer” or “FIP” as used herein refers to an oligonucleotide having a 3′ end and a 5′ end, wherein the oligonucleotide comprises a first part at the 3′ end of the oligonucleotide and a second part at the 5′ end of the oligonucleotide. Generally, the first part is complementary to a nucleotide sequence in the antisense strand of the target polynucleotide and the second part is complementary to a nucleotide sequence in the sense strand of the target polynucleotide. In other words, the first part may be a F2 region and the second part may be an F1c region. The F2 region is complementary to the F2c region in the target DNA sequence while the F1c region is complementary to the F1 region in the target DNA sequence (FIG. 1).

The term “backward inner primer” or “BIP” as used herein refers to an oligonucleotide having a 3′ end and a 5′ end, wherein the oligonucleotide comprises a first part at the 3′ end of the oligonucleotide and a second part at the 5′ end of the oligonucleotide. Generally, the first part is complementary to a nucleotide sequence in the sense stand of the target polynucleotide and the second part is complementary to a nucleotide sequence in the antisense strand of the target polynucleotide. In other words, the first part may be a B2 region and the second part may be a B1c region. The B2 region is complementary to the B2c region in the target DNA sequence while the B1c region is complementary to the B1 region in the target DNA sequence (FIG. 1).

The term “forward outer primer” or “F3” as used herein refers to an oligonucleotide, which is complementary to a nucleotide sequence in the antisense stand of the target polynucleotide. Thus, F3 may comprise a region that is complementary to a F3c region in the target DNA sequence (FIG. 1).

The term “backward outer primer” or “B3” as used herein refers to an oligonucleotide which is complementary to a nucleotide sequence in the sense strand of the target polynucleotide. Thus, B3 may comprise a region that is complementary to a B3c region in the target DNA sequence (FIG. 1).

The term “forward loop primer” or “FL” as used herein refers to an oligonucleotide that is complementary to a polynucleotide sequence in the sense strand of the target polynucleotide. Thus FL is complementary to a section on the target DNA sequence designated FLc (FIG. 1).

The term “backward loop primer” or “BL” as used herein refers to an oligonucleotide that is complementary to a polynucleotide sequence in the antisense strand of the target polynucleotide. Thus, BL is complementary to a section on the target DNA sequence designated BLc (FIG. 1).

The term “nucleic acid amplification” as used herein refers to any nucleic acid step/method/protocol that is used to replicate and multiply a particular nucleic acid sequence in a sample.

The term “LAMP” refers to loop-mediated isothermal amplification. LAMP is a reaction for amplification of nucleic acids. LAMP uses 4 (or 6) primers targeting 6 (or 8) regions within or surrounding the target sequence (FIG. 1). The method relies on isothermal conditions, i.e. it is carried out at a constant temperature and does not require a thermal cycler. Briefly, LAMP amplification of the target gene occurs when it is incubated with the target specific primers FIP, BIP, F3 and B3, and a polymerase at a constant temperature in the range of 59 and 75° C. Inclusion of forward and backward loop primers, FL and BL respectively, may be added to the LAMP amplification. The amplification product can be detected by any appropriate method. The outcome of the method can be correlated with the number of DNA copies produced during LAMP amplification and thus serve as the basis for quantification of the amount of DNA present in the sample. Quantitative detection can be performed both by end-point and real-time measurements. The DNA may also be cDNA:

The term “RT-LAMP” refers to reverse transcription loop-mediated isothermal amplification. RT-LAMP combines LAMP with a reverse transcription step to allow the detection of RNA.

The term “nucleic acid amplification reagents” as used herein refers to agents which are added to a sample in order to effect amplification of any target polynucleotide(s) within the sample.

The term “LAMP reagents” as used herein refers to agents, which are added to a LAMP in addition to a sample and at least four primers. The LAMP reagents comprise at least nucleotides and a nucleic acid polymerase. In addition, the LAMP reagents may comprise other compounds such as salt(s) and buffer(s). The nucleic acid polymerase preferably has a high strand displacement activity and replication activity.

The term “signaling substance” as used herein refers to any physical quantity of the amplification reagents (such as LAMP reagents) or sample which undergoes change, or an agent produced, consumed or undergoing a phase change during nucleic acid amplification and the amount of which can be detected and thus used to demonstrate or quantify the amplification reaction, such as a production of H+-ions, or a drop in pH or conductivity of the sample/reaction mixture.

The term “target sequence” as used herein refers to any nucleic acid sequence, which is desired to detect. In an embodiment, the target sequence is a nucleic acid sequence, preferably a nucleic acid sequences which can be amplified by any nucleic acid amplification technology, such as LAMP, preferably wherein the target nucleic acid sequence is amplified using at least four primers flanking the target sequence.

The term “target polynucleotide” as used herein encompasses the term “target nucleic acid” and the two definitions may be used interchangeably unless expressly stated otherwise.

The term “electrochemical device” as used herein refers to any device capable of study an analyte by measuring the potential (volts) and/or current (amperes) in an electrochemical cell containing the analyte. Typically, the three main categories for measuring using an electrochemical device are potentiometry (the difference in electrode potentials is measured), coulometry (the cell's current is measured over time), and voltammetry (the cell's current is measured while actively altering the cell's potential). The term “electrochemical device” as used herein may also be used interchangeably with the term “three-electrode electrochemical cell” when referring to the first aspect of the invention. For the avoidance of doubt, unless expressly stated to the contrary, all embodiments as described herein with reference to the “electrochemical device” refer to both the electrochemical device of the second aspect of the invention and the three-electrode electrochemical cell of the first aspect of the invention.

The term “three-electrode electrochemical cell” as used herein refers to an electrochemical cell comprising three different electrodes, being a working electrode, a counter electrode and a reference electrode. In use, all three electrodes of the electrochemical cell are in contact with the solution being analysed. During three-electrode experiments, charge flow (current) primarily occurs between the working electrode and the counter electrode while the potential of the working electrode is measured with respect to the reference electrode.

The term “two-electrode electrochemical cell” as used herein refers to an electrochemical cell comprising two electrodes, wherein one electrode is a working electrode and the second electrode is a combined reference and counter electrode. That is to say, the second electrode is a variation of a three-electrode cell wherein the reference and counter electrode are shorted and act as one electrode, i.e. they are the same electrode.

The term “electrode” as used herein refers to an electrically conductive material (often made from carbon, metal, or a composite) that provides a path for current to flow into, or out of, an electrochemical system. During an electrochemical measurement, some portion of the electrode surface is in direct contact with an ionically-conductive medium, or electrolyte, through which charge is transferred between other electrodes.

By the term “quinone” as used herein, this refers to organic compounds that are cyclic organic compounds containing two carbonyl (C═O) groups either adjacent or separated by a vinylene (—CH═CH—) group.

By the term “quinone derivative” as used herein, this refers to compounds that are derived from quinones as defined above wherein one or more of the hydrogen atoms in the cyclic backbone of the compound has been replaced by other atoms or radicals.

As used herein, the term “squarewave voltammetry” refers to a form of linear potential sweep voltammetry that uses a combined square wave and staircase potential applied to a stationary electrode.

As used herein, the term “electrochemical response” refers to any measurable process that either causes or is accompanied by the passage of an electric current. Such measurable electrochemical responses include measuring the potential, the current, or the impedance of a system, or indeed a combination of any of these measurements.

The term “pH indicator” as used herein refers to a halochromic chemical compound, which is a chemical compound that changes colour when pH changes occur, and which, if added in small amounts to a solution, allow the pH (acidity or basicity) of the solution to be determined visually.

The term catechol, as used herein, comprises all groups having a structural motive according to the following general formula I:

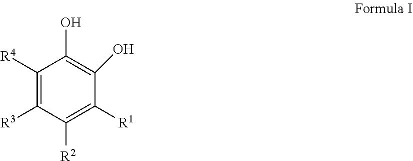

Thereby, residues R¹, R², R³ and R⁴ can be absent, hydrogen or any organic or organometallic residue. Preferred residues are aliphatic or aromatic chains (such as, e.g., C₁-C₁₀ aliphatic chains or C₆-C₂₀ aromatic residues) that can optionally be interrupted or substituted by moieties containing nitrogen, oxygen and/or sulfur atoms, e.g., by —NH₂, —NH—, —OH, ═O, —O—, —SH and/or —S—S—. Residues R¹, R², R³ and R⁴ can have independently from each other the same meaning or different meanings in each case. The term catechol, as used herein, also refers to a substituted ortho-dihydroxybenezene derivative. Two different isomeric conformations are represented by Catechol is also known as pyrocatechol and benzene-1,2-diol.

The term “catecholic group” also refers to the oxidized form of catechol and its derivatives that is also known as quinone and corresponds to the following general formula II:

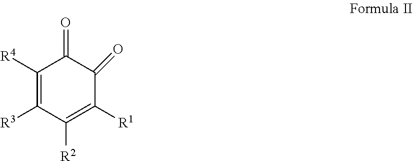

The residues R¹, R², R³ and R⁴ can have the same meaning as previously explained.

The term “receptacle” as used herein relates to a vessel such as a container suitable for accommodating a liquid, such as an aqueous solution. A receptacle usually comprises an aperture and a lid that can be used to close that aperture. A receptacle according to the present disclosure may also comprise three-electrode electrochemical cell.

Method of Measuring the pH of a Solution

According to one aspect of the invention there is provided a method of measuring the pH of a solution, wherein the method comprises the steps of:
providing a solution comprising a compound capable of undergoing a change in its oxidation state and/or structural conformation as a function of the pH of the solution;
applying the solution to a two or three-electrode electrochemical cell;
measuring an electrochemical response of the electrochemical cell; and
quantifying the pH of the solution as a function of the electrochemical response of the electrochemical cell.

According to another aspect of the invention there is provided a method of measuring the pH of a solution, wherein the method comprises the steps of:
providing a solution comprising a quinone, a quinone derivative, and/or a pH indicator;
applying the solution to a three-electrode electrochemical cell;
measuring an electrochemical response of the electrochemical cell; and
quantifying the pH of the solution as a function of the electrochemical response of the electrochemical cell.

In an embodiment, the solution comprises a plurality of compounds capable of undergoing a change in their oxidation state and/or structural conformation as a function of the pH of the solution. That is to say that the solution may comprise a plurality of structurally different compounds capable of undergoing a change in their oxidation state and/or structural conformation as a function of the pH of the solution, such as two or more compounds, for example three, four, five, six, seven, eight, nine or ten or more compounds.

In another embodiment the compound capable of undergoing a change in its oxidation state and/or structural conformation as a function of the pH of the solution is a catechol, a catechol derivative and/or a compound comprising a catecholic group.

In another embodiment the compound capable of undergoing a change in its oxidation state and/or structural conformation as a function of the pH of the solution is a quinone, a quinone derivative, a pH indicator, or combinations thereof.

In a further embodiment the compound capable of undergoing a change in its oxidation state and/or structural conformation as a function of the pH of the solution is a quinone or a quinone derivative selected from the list consisting of 1,2-benzoquinone, 1,4-benzoquinone, 1,4-naphthoquinone, 9,10-anthraquinone, and derivatives and combinations thereof.

In another embodiment the compound capable of undergoing a change in its oxidation state and/or structural conformation as a function of the pH of the solution is a pH indicator selected from the list consisting of malachite green oxalate, brilliant green, eosin yellowish, erythrosine B, methyl green, methyl violet, picric acid, cresol red, crystal violet, m-Cresol purple, thymol blue, p-Xylenol blue, Eosin (bluish), quinaldine red, 2,4-dinitro phenol, 4-(dimethylamino) azobenzol, bromochlorophenol blue, bromophenol blue, congo red, methyl orange, bromocresol green, 2,5-dinitrophenol, alizarin sulphonic acid, methyl red, chlorophenol red, litmus, bromocresol purple, bromophenol red, 4-nitrophenol, bromoxylenol blue, bromothymol blue, phenol red, 3-nitrophenol, neutral red, cresol red, 1-naphtholphthalein, m-cresol purple, thymol blue, p-xylenol blue, phenolphthalein, thymolphthalein, alkali blue, alizarin yellow GG, indigo carmine, epsilon blue, titan yellow, and combinations thereof. In an embodiment, the solution comprises at least two of the compounds listed in this paragraph, for example at least three, four or five compounds listed in this paragraph.

In one embodiment of the present disclosure, two or more compounds, said compounds being a catechol, a catechol derivative, a compound comprising a catechol group, a quinone, a quinone derivative or a pH indicator, are provided in the same aqueous solution. Thus, pH measurements and quantification can be conducted on a larger pH scale, such as between pH 5 and 8, such as between pH 4 and 8, such as between pH 3 and 8, such as between pH 3 and 9, such as between pH 4 and 9, such as between pH 5 and 9, such as between pH 6 and 9, such as across all pH range.

In one embodiment of the present disclosure, the three-electrode electrochemical cell does not comprise a quinhydrone electrode. In fact, a quinhydrone electrode cannot effectively measure pH of solutions where the test solution itself causes a change in open circuit potential (OCP) over time due to biochemical or chemical reactions. Further, quinhydrone electrodes suffer from a salt error. The functioning of quinhydrone electrode may be impaired by the presence of oxidizing and reducing agents and the quinhydrone electrode is poisoned by traces of metals such as copper, silver, and other metals below antimony in the electromotive series, which may also interferer with pH measurements if complexing agents are present in the solution to be analyzed.

More importantly, a quinhydrone electrode is not suitable for usage in a three-electrode electrochemical cell, because quinhydrone and its aqueous dissociation products cannot be electrochemically analyzed, such as distinguished, in a three-electrode electrochemical cell. Thus, a quinhydrone electrode is not suitable for use in the methods and systems disclosed herein.

Hence, in one embodiment of the present disclosure the quinone, quinone derivative, and/or pH indicator is not quinhydrone.

Using a catechol, a catechol derivative and/or a compound comprising a catecholic group, such as a quinone or quinone derivative, such as any one of the listed specific compounds, for example phenol red, provides several advantages with quantification of the pH of a solution. One of the advantages is a high resolution in the pH measurement, in particular a higher resolution compared to quantifying pH with a standard pH meter that measures hydrogen-ion activity in aqueous solutions. This is due to the rich redox chemistry that characterizes a catechol, a catechol derivative and/or a compound comprising a catecholic group, such as a quinone or quinone derivative, such as any one of the listed specific compounds, for example phenol red, as provided in detail in Example 3 of the present disclosure.

Thus, the method and system disclosed in the present disclosure provide accurate and instantaneous pH measurements as the response time of the pH measurement is not dependent on the diffusion of ions, for example protons, across a semi-permeable membrane or an adsorbing membrane, but instead based on measuring an electrochemical response that correlates with the electrochemical state of a catechol, a catechol derivative and/or a compound comprising a catecholic group, such as a quinone or quinone derivative, or a pH indicator.

In a further embodiment the compound capable of undergoing a change in its oxidation state and/or structural conformation as a function of the pH of the solution is phenol red.

In another embodiment the compound capable of undergoing a change in its oxidation state and/or structural conformation as a function of the pH of the solution is a weak acid, for example a weak acid having a pKa of from about 2 to about 14, such as a pKa from about 4 to about 12, for example a pKa of from about 6 to about 10.

In an embodiment the compound capable of undergoing a change in its oxidation state and/or structural conformation as a function of the pH of the solution is dissolved in the solution. That is to say, the compound capable of undergoing a change in its oxidation state and/or structural conformation as a function of the pH of the sample solution is not bound, in particular not irreversibly bound, either chemically or through any physical means, to any of the electrodes of the two or three-electrode chemical cell.

In another embodiment the solution is an aqueous solution. In particular, the solution is an aqueous solution comprising at least 90 vol. % water, such as at least 91, 92, 93, 94, 95, 96, 97, 98 or 99 vol. % water. For example, the solution is an aqueous solution comprising greater than 99 vol. % water.

In an embodiment the potential of the two or three-electrode electrochemical cell is measured via potentiodynamic electrochemistry, such as cyclic squarewave voltammetry, squarewave voltammetry, linear sweep voltammetry, cyclic voltammetry or open circuit potentiommetry.

In a further embodiment, the electrochemical cell is a two-electrode chemical cell and the potential of the cell is measured by open circuit potentiometry.

In an embodiment, the electrochemical cell is a three-electrode chemical cell and the potential of the cell is measured by cyclic squarewave voltammetry, squarewave voltammetry, linear sweep voltammetry, or cyclic voltammetry.

In another embodiment, the squarewave voltammetry step is run at a scan range of about −2 to about 2 V vs. Ag/AgCl, such as from about −1 to about 1 V vs. Ag/AgCl, for example from about −0.6 to about 0.6 V vs. Ag/AgCl.

In a further embodiment, the squarewave voltammetry step is run at a frequency of about 1 to about 50 Hz, such as from about 5 to about 20 Hz.

In an embodiment, the squarewave voltammetry step is run at a potential step of from about 1 to about 20 mV, such as from about 1 to about 10 mV.

In another embodiment, the squarewave voltammetry step is run at an amplitude of from about 1 to about 50 mV, such as from about 1 to about 25 mV, for example from about 5 to about 20 mV.

In a further embodiment the concentration of the compound capable of undergoing a change in its oxidation state and/or structural conformation as a function of the pH of the solution within the solution is from about 1 μM to about 1000 μM, such as from about 1 μM to about 500 μM, for example from about 5 μM to about 250 μM, in particular from about 50 μM to about 200 μM, for example from about 50 μM to about 150 μM, such as from about 50 μM to about 100 μM.

In an embodiment the step of quantifying the pH of the solution as a function of the electrochemical response of the electrochemical cell is performed via application of a regression algorithm, such as a linear regression algorithm. Without wishing to be bound by theory, the linear regression algorithm correlates the pH with the function of redox potential and current in a linear fashion.

In another embodiment the solution comprises a buffer.

In an embodiment, the buffer comprises trisaminomethane in an amount of from 20 to 450 μM.

In a further embodiment, the buffer comprises (NH₄)₂SO₄ in an amount of from 5 to 20 mM.

In another embodiment, the buffer comprises KCl in an amount of from 25 to 100 mM.

In an embodiment, the buffer comprises MgSO₄ in an amount of from 5 to 20 mM.

In an embodiment, the buffer comprises deoxynucleoside triphosphate in an amount of from 1 to 5 mM.

In another embodiment, the buffer comprises tween-20 in an amount of from 0.05 to 1% v/v.

Another aspect of the present disclosure relates to a use of a quinone, a quinone derivative, and/or a pH indicator, for quantifying the pH of a solution, wherein the solution is in an electrochemical cell, and wherein the pH is quantified by measuring an electrochemical response in the electrochemical cell.

Methods of Quantifying a Target Polynucleotide

As defined further below, the method of the first aspect of the invention may also comprise a step of providing a sample. In an embodiment, the sample is provided in the solution, that is to say the solution may comprise the sample. The sample may be any sample. In a preferred embodiment, the sample is a biological sample. None limiting examples hereof are body fluid samples or tissue samples as defined further below.

It is envisaged that the sample may or may not comprise a target polynucleotide/target nucleic acid. Therefore, in an embodiment of the first aspect of the invention, the method allows for determining the presence of a target polynucleotide in the solution. That is to say, the method allows for measuring the pH of the solution and from this allows for determining whether or not a target polynucleotide is present in the solution.

In an embodiment the solution further comprises primers and nucleic acid amplification reagents, preferably wherein the nucleic acid amplification reagents are LAMP reagents.

In a further embodiment the nucleic acid amplification reagents comprise a signaling substance, or are capable of releasing a signaling substance, or comprise both a signaling substance and a substance capable of release a signaling substance.

In another embodiment the signaling substance is H⁺ and/or H₃O⁺.

In another embodiment the LAMP reagents comprise a reverse transcriptase.

In a further embodiment the primers comprise a forward inner primer, a backward inner primer, a forward outer primer and a backward outer primer.

In an embodiment the LAMP amplification is performed with an additional pair of loop primers, such as forward loop primer and/or backward loop primer.

In an embodiment the FIP/BIP is present in the solution in an amount of 1.6 μM, the F3/B3 is present in an amount of 0.2 μM, and the LoopF/B is present in an amount of 0.4 μM.

In an embodiment Bst DNA polymerase is present in the solution in an amount of 0.32 U/μL and reverse transcriptase is present in an amount of 0.3 U/μL.

In an embodiment the method comprises a step of performing a nucleic acid amplification step (e.g. an isothermal nucleic acid amplification step), preferably a plurality of nucleic acid amplification steps. The nucleic acid amplification step(s) is/are advantageously performed after the solution is applied to the two or three-electrode electrochemical cell. In a further embodiment, the steps of performing the nucleic acid amplifications, measuring the electrochemical response of the electrochemical cell and quantifying the pH of the solution as a function of the electrochemical response of the electrochemical cell may be performed sequentially in this order, or in any other order, or may be performed simultaneously, and the steps may also be performed simultaneously and continuously.

It is envisaged that any nucleic acid amplification step/protocol/method may be used in the method of the first aspect of the invention and such amplification methods may be selected from the list consisting of polymerase chain reaction (PCR) (including nested (n), quantitative (q) or real-time reverse transcriptase (RT) PCR), LAMP as defined elsewhere herein, Rolling Circle Amplification, and quantitative nucleic acid sequence-based amplification (QT-NASBA).

In a particular embodiment the nucleic acid amplification step/protocol/method is isothermal, such as wherein the nucleic acid amplification is isothermal and carried out at a temperature in the range of from 40 to 80° C., such as from 50 to 70° C., for example from 57 to 65° C.

In an embodiment, the nucleic acid isothermal amplification step is a LAMP step or a Rolling Circle Amplification step.

In a further embodiment the nucleic acid amplification step(s) is/are a LAMP step. For the avoidance of doubt, where only one nucleic acid amplification step is performed then in this embodiment only one LAMP step is performed. However, when multiple nucleic acid amplification steps are envisaged then multiple LAMP steps are performed.

In an embodiment the method is for quantifying the amount of a target polynucleotide in the solution or sample provided in the solution.

In another embodiment the step of measuring the electrochemical response of the electrochemical cell comprises measuring a change in the current and/or impedance and/or potential of the electrochemical cell due to a concentration change of the signaling substance.

In a further embodiment the method comprises a step of deriving an amplification rate of the nucleic acid from the rate of the change of the electrochemical response (e.g., current and/or impedance and/or potential) of the two or three-electrode electrochemical cell.

In an embodiment the step of deriving the amplification rate of the nucleic acid is performed simultaneously to the step of measuring the electrochemical response of the two or three-electrode electrochemical cell.

In a further embodiment the nucleic acid amplification step produces or consumes a signaling substance, such as H⁺, as the nucleic acid is amplified. This is on the proviso that a nucleic acid/polynucleotide is present in the solution in the first instance. The production or consumption of the signaling substance in turn causes a change in the current and/or potential and/or impedance of the electrochemical cell due to a concentration change in the signaling substance. As a result of the change in the current and/or potential and/or impedance of the electrochemical cell, the pH value quantified also changes and from this change in pH an amplification rate of the nucleic acid/target polynucleotide may be derived. For the avoidance of doubt, the step of deriving an amplification rate is not necessary in order to determine whether or not a target polynucleotide is present in the solution as any detectable change in the current and/or potential and/or impedance of the electrochemical cell and subsequently quantified pH change would indicate the presence of the polynucleotide.

Therefore, a particular first aspect of the invention provides a method of measuring the pH of a solution and of determining the presence of a target polynucleotide in the solution, wherein the method comprises the steps of:
a. providing a solution comprising a compound capable of undergoing a change in its oxidation state or structural conformation as a function of the pH of the solution, and a sample;
b. applying the solution to a two or three-electrode electrochemical cell;
c. performing at least one, or a plurality of, nucleic acid amplification steps wherein the nucleic acid amplification(s) produce or consume a signaling substance, if a target polynucleotide is present;
d. measuring the electrochemical response of the electrochemical cell, such as the change in the current, and/or potential, and/or impedance of the electrochemical cell, due to a concentration change of the signaling substance;
e. quantifying the pH of the solution and/or the change in the pH of the solution as a function of the electrochemical response of the electrochemical cell; and
f. optionally deriving the amplification rate of a target polynucleotide in the sample, if originally present, by the rate of the change of the electrochemical response of the electrochemical cell and thereby quantifying the amount of target polynucleotide in the sample.

Therefore, a particular first aspect of the invention provides a method of measuring the pH of a solution and of determining the presence of a target polynucleotide in the solution, wherein the method comprises the steps of:
a. providing a quinone, a quinone derivative, and/or a pH indicator;
b. applying the solution to a three-electrode electrochemical cell;
c. performing at least one, or a plurality of, nucleic acid amplification steps wherein the nucleic acid amplification(s) produce or consume a signaling substance, if a target polynucleotide is present;
d. measuring the electrochemical response of the electrochemical cell, such as the change in the current, and/or potential, and/or impedance of the electrochemical cell, due to a concentration change of the signaling substance;
e. quantifying the pH of the solution and/or the change in the pH of the solution as a function of the electrochemical response of the electrochemical cell; and
f. optionally deriving the amplification rate of a target polynucleotide in the sample, if originally present, by the rate of the change of the electrochemical response of the electrochemical cell and thereby quantifying the amount of target polynucleotide in the sample.

Steps c., d., e. and f. in the above paragraphs may be performed individually either sequentially in the ordered indicated, or in another order, or they may be performed simultaneously, for example simultaneously and continuously.

For the avoidance of doubt, the invention is not envisaged to be limited to whether or not a target polynucleotide is originally present in the sample. That is to say, the nucleic acid amplification step may be performed on a solution containing a sample that does not comprise a target polynucleotide and in this scenario this would result in zero, or at least a non-detectable, change in the electrochemical response (e.g. the current and/or potential and/or impedance) of the electrochemical cell, leading to a negative result for the target polynucleotide.

Target Polynucleotide

The target polynucleotide may be any polynucleotide sequence. The target polynucleotide may in particular be a viral or bacterial polynucleotide. The target polynucleotide is ideally a stretch of DNA that is unique to a virus type or bacterial species and strand, and at the same time evolutionarily conserved enough to be stable in the genome of the particular virus or bacterium.

In one embodiment, the virus is selected from the group consisting of dsDNA viruses, ssDNA viruses, dsRNA viruses, (+)ssRNA viruses RNA, (−)ssRNA viruses RNA, ssRNA-RT viruses RNA and dsDNA-RT viruses DNA.

In a preferred embodiment the virus an (+)ssRNA virus RNA.

In one embodiment, the polynucleotide may be any polynucleotide present in an influenza A virus, influenza B virus, Zika virus, SARS-CoV virus, or MERS-CoV virus.

In one embodiment, the polynucleotide may be any polynucleotide present in a corona virus.

In one embodiment, the target polynucleotide may be any polynucleotide present within the corona virus, such as SARS-CoV-2 RNA.

In one embodiment the target polynucleotide sequence is a sequence of SEQ ID NO:1 or SEQ ID NO:2.

In one embodiment the target polynucleotide sequence comprises 70.0 percent, 71.0 percent, 72.0 percent, 73.0 percent, 74.0 percent, 75.0 percent, 76.0 percent, 77.0 percent, 78.0 percent, 79.0 percent, 80.0 percent, 81.0 percent, 82.0 percent, 83.0 percent, 84.0 percent, 85.0 percent, 86.0 percent, 87.0 percent, 88.0 percent, 89.0 percent, 90.0 percent, 91.0 percent, 92.0 percent, 93.0 percent, 94.0 percent, 95.0 percent, 96.0 percent, 97.0 percent, 98.0 percent, 99.0 percent, and 100.0 percent homology or sequence identity to SEQ ID NO:1 or SEQ ID NO:2.

The percent sequence identity (or homology) between two polypeptides may be determined using suitable computer programs, for example the GAP program of the University of Wisconsin Genetic Computing Group and it will be appreciated that percent identity is calculated in relation to polypeptides whose sequence has been aligned optimally. The alignment may alternatively be carried out using the Clustal W program (Thompson et al., (1994) Nucleic Acids Res 22, 4673-80). The parameters used may be as follows: Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

Sample

In the second aspect of the invention, the method comprises a step of providing a sample. The sample may be any sample. In a preferred embodiment, the sample is a biological sample. None limiting examples hereof are body fluid samples or tissue samples.

In an even more preferred embodiment the sample comprises human, animal plant, bacterial, fungal, or protozoan cells.

In the first aspect of the invention, the method may also comprise a step of providing a sample. In an embodiment, the sample is provided in the solution, that is to say the solution may comprise the sample. The sample may be any sample. In a preferred embodiment, the sample is a biological sample. None limiting examples hereof are body fluid samples or tissue samples.

The sample may comprise a target polynucleotide as defined above. Specifically, the target polynucleotide may be any polynucleotide sequence/nucleic acid sequence. The target polynucleotide in the sample may in particular be a viral or bacterial polynucleotide. The target polynucleotide in the sample may be a stretch of DNA that is unique to a virus type or bacterial species and strand, and at the same time evolutionarily conserved enough to be stable in the genome of the particular virus or bacterium.

In an embodiment, the sample may be an isolated DNA sample, an isolated RNA sample, a crude DNA extract sample or a crude RNA extract sample.

In one embodiment, the sample is a nasal sample, a throat sample, an anal sample, a vaginal sample, an ear draining sample, a skin surface swab sample, a urine sample, a whole blood sample, a serum sample, a plasma sample or lymph drainage sample.

In another embodiment, the sample is a tissue sample. The tissue sample may be any tissue sample, such as an epithelial tissue sample, connective tissue sample, muscle tissue sample and/or nervous tissue sample.

The methods may furthermore comprise one step of preparing the sample before applying the sample to the electrochemical device/two or three-electrode electrochemical cell. The sample may be prepared by any suitable method. Examples hereof are described in Example 1 herein below.

Generally, the sample may be mixed with any suitable medium. A person skilled in the art would know a suitable medium for a specific samples. The medium may be a transport medium. The sample and transport medium can be added directly to the electrochemical device, or heated prior to applying the sample and the medium at any suitable temperature for a suitable amount of time.

In one embodiment, the sample is inoculated in transport medium. A transport medium may be any suitable medium for transporting the target polynucleotide sequence. In a preferred embodiment, the transport medium is a viral transport medium.

In another embodiment, the sample is mixed with an anticoagulant like heparin, EDTA or sodium citrate.

The sample, optionally mixed with a medium, may be lysed, for example by mechanical lysis, thermal lysis, acoustic cavitation, osmotic shock, enzymatic lysis or chemical lysis.

In one embodiment, the sample is mixed with the at least four primers and LAMP reagents prior to applying the sample to the electrochemical device.

In one embodiment, the sample is heated at 60-100° C., for example at 65-95° C., for example at 70-90° C., for example at 75-85° C.

In one embodiment, the sample is heated for at least 1 minute, such as at least 2 minutes, such as at least 5 minutes.

In one embodiment, the methods of the present invention are capable of detecting whether a target polynucleotide is present in the sample.

In another embodiment, the methods of the present invention are capable of quantifying the amount of target polynucleotide in a sample.

Electrolytical Device

In the first aspect of the invention, the method comprises the step of adding the solution to a two or three-electrode electrochemical cell.

In the second aspect of the invention, the method comprises a step of providing an electrochemical device comprising an electrochemical cell having multiple film electrodes.

In both aspects of the invention, the electrochemical device/two or three-electrode electrochemical cell may be a planar device with film electrodes positioned in a single layer. Thereby, the film electrodes may be patterned, onto a planar substrate, with a thickness of below 2 mm, such as by screen printing or thick film technology. Screen-printed electrodes (SPE) are commonly used for forming disposable and low-cost sensors and biosensors. The electrodes of such a sensor can be manufactured by the use of a wide range of conductive inks, and in various shapes and dimensions, depending on the analytical needs. Besides their fabrication, these platforms are suitable to be customized with a variety of materials, including nanomaterials, and bio elements. Consequently, in one embodiment of the present disclosure, the electrodes are screen printed electrodes, preferably printed on a planar substrate of an inert material.

In one embodiment of the present disclosure, the electrodes of the three-electrode electrochemical cell are wire electrodes.

Preferably, the area of the electrochemical device/two or three-electrode electrochemical cell is smaller than 500 mm², yet more preferably smaller than 300 mm², making the electrochemical device/two or three-electrode electrochemical cell portable and suitable for a point-of-care setting. The electrodes of the electrochemical device/two or three-electrode electrochemical cell may extend towards an edge of the planar substrate, and thereby form an edge connection, for electrical connection to a measurement setup, such as a potentiostat. The potentiostat may itself be a portable potentiostat, such as a hand-held potentiostat.

The electrochemical device in the second aspect of the invention may comprise multiple film electrodes, for example a working electrode, a reference electrode and a counter electrode, wherein the electrodes preferable forms a part of a three-electrode system, such as a electrochemical three-electrode system.

A working electrode of a three-electrode system is typically the electrode at which the redox process of interest occurs. Thereby, the focus of an electroanalytical measurement is typically on a particular electrochemical reaction occurring at the working electrode.

A reference electrode typically has a stable and well-known thermodynamic potential. The high stability of the reference electrode is usually achieved by employing a redox system with constant (buffered or saturated) concentrations of the ions or molecules involved in the redox half-reaction. When used as part of a three-electrode system, current does not pass through the reference electrode.

A counter electrode, also called the auxiliary electrode, is typically used in an electrochemical system to complete the electrical circuit with the working electrode. While the redox process of interest typically occurs on the working electrode, the counter electrode may serve as a source or depository of current so as not to limit the electrochemical reactions at the working electrode.

The electrodes of the electrochemical device/two or three-electrode electrochemical cell may be fabricated in a suitable material, such as gold, silver, carbon, platinum, ruthenium dioxide, or a combination thereof. The material of the electrodes may be selected individually, thereby the material of a number of the electrodes, such as each electrode, may be different. Alternatively, the electrodes may be provided in the same material. However, for providing simplicity, stability, and a capability of miniaturization, it may be a preference that the material of the reference electrode and the counter electrode is Ag/AgCl. In an alternative embodiment of the present disclosure, the material of the electrodes may be identical.

Additionally, any, or all, of the electrodes may comprise an ion-selective membrane, such an electrode may be referred to as an ion-selective electrode. Said membrane may be used to convert the activity of a specific ion dissolved in a sample into an electrical potential. The membrane may cover at least a part of the electrode, and located such that it forms an interface to a solution during a measurement. The membrane is preferably a glass membrane, typically an ion-exchange type of glass (silicate or chalcogenide). Alternatively, the membrane may be a crystalline membrane, an ion-exchange resin membrane or an enzyme membrane.

Alternatively, or additionally, to the use of an ion-selective membrane, the surface of any of the electrodes may be modified. For example, the surface of the working electrode may be chemically modified to allow for binding of hydroxide OH⁻ and/or hydronium H₃O⁺ ions. In an embodiment of the present disclosure, the surface of any of the electrodes may comprise a pH sensing layer of a solid-state membrane with combination of Cobalt

Oxide and Iridium Oxide. Preferably, the surface of the working electrode comprises a pH sensing layer of a solid-state membrane with combination of Cobalt Oxide and Iridium Oxide.

Further modifications of the electrode surfaces include chemical modification by, ruthenium oxide, graphene platelets, exposed thiol group and/or exposed hydroxyl groups.

Preferably, the surface of any of the electrodes, for forming contact with the sample/solution, has a fixed concentration of chloride ions, such as for a true reference electrode measurement. Thereby, any, or all of the electrodes may comprise a surface having a fixed concentration of chloride ions.

In a preferred embodiment of the present disclosure, the material of the working electrode is carbon, and the material of the counter electrode and the reference electrode is Ag/AgCl.

The electrochemical device/two or three-electrode electrochemical cell may be formed by screen printing of electrodes on a planar substrate. Thereby, the electrodes may be screen printed electrodes wherein the electrodes are in a single layer. The material of the planar substrate is preferably a chemically inert material, such as alumina, ceramic, glass, or an inert plastic. The electrochemical device/two or three-electrode electrochemical cell preferably has a small form-factor, making it portable, and suitable for point-of-care measurements.

It is a preference that the electrochemical device/two or three-electrode electrochemical cell comprises an inlet for receiving the solution to be analyzed. The inlet may be a microfluidic inlet and may further be connected to the electrochemical cell by a hydrophilic zone. The hydrophilic zone is ideally configured such that a solution provided to the microfluidic inlet is transported, by capillary action, to the electrochemical cell. Alternative means of storing and providing a solution to the electrochemical cell comprise the use of a microwell superpositioned on the electrochemical cell. The microwell may be bonded to the electrochemical device, and/or by lithography.

When the solution is provided to the electrochemical cell, it may be an advantage to apply a vapour barrier for preventing evaporation of the solution. This may especially be a requirement during longer measurement, such as at least several minutes. The vapour barrier may be formed by fluidly sealing the inlet of the electrochemical device, or the microwell, with a physical lid, a heated lid, a mineral oil, and/or a paraffin wax. Thereby, the vapour barrier act to form a fluidly sealed device comprising the solution.

In an embodiment of the present disclosure, the electrochemical device may comprise the primers and the LAMP reagents. In other words, the primers and LAMP reagents may be preloaded to the device prior to loading of the sample/solution.

In one aspect of the present disclosure, there is provided a system for measuring the pH of a solution, the system comprising:
a. An instrument comprising a potentiostat; and
   iii. A first receptacle comprising a three-electrode electrochemical cell, and a second receptacle comprising a quinone, a quinone derivative, and/or a pH indicator, or
   iv. A first receptacle comprising a three-electrode electrochemical cell and a quinone, a quinone derivative, and/or a pH indicator,
   wherein the potentiostat is configured to measure an electrochemical response of the electrochemical cell.

In one embodiment, the quinone, quinone derivative, and/or pH indicator of the system disclosed herein are dissolved in an aqueous solution in the first or in the second receptacle.

In one embodiment, the quinone, quinone derivative, and/or pH indicator of the system disclosed herein are dissolved in an aqueous solution in the first receptacle, wherein said aqueous solution also comprises a nucleic acid amplification reaction system, such as at least two primers configured to flank a target sequence and LAMP reagents.

In one embodiment, the quinone, quinone derivative, and/or pH indicator of the system disclosed herein are dissolved in an aqueous solution in the second receptacle, whereas the first receptacle comprises another aqueous solution. Thus, prior to measuring the pH of the aqueous solution in the first receptacle, a suitable volume of the aqueous solution comprising the quinone, quinone derivative, and/or pH indicator is transferred to the first receptacle.

In one embodiment, the potentiostat is configured to measure an electrochemical response of the electrochemical cell.

In one embodiment, the potentiostat is configured to measure an electrochemical response, wherein the electrochemical response is representative of the oxidation state of the quinone, quinone derivative, and/or pH indicator in the first receptacle.

In one embodiment, the system further comprises a heating unit, configured for heating the first and/or second receptacle, such as to a temperature within the in the range of from 59° C. to 75° C.

In one embodiment, the first receptacle accommodates a nucleic acid amplification reaction system, such as at least two, or at least four primers configured to flank a target polynucleotide sequence and LAMP reagents.

In one embodiment, the first receptacle comprises a microfluidic inlet for receiving a solution and/or a sample.

In one embodiment, the first and/or the second receptacles are replaced by new first and/or the second receptacles after each use.

In one embodiment, the microfluidic inlet is connected to the electrochemical cell by a hydrophilic zone, such as a porous/fibrous structure or a hydrophilic channel, configured to transport the sample by capillary action.

In one embodiment, the first receptacle and/or the second receptacle is an Eppendorf tube, a microwell, or a culture flask.

In one embodiment, the electrodes in the system of the present disclosure are as defined herein.

LAMP Amplification

The methods of the second aspect of the invention comprise a step of performing a plurality of LAMP amplifications each comprising the sample (for example provided and/or prepared as described in the “Sample” section herein above) thereby amplifying the target polynucleotide sequence. Each LAMP amplification comprises at least four primers each set flanking the target sequence and LAMP reagents. The LAMP amplification is performed under isothermal conditions.

In an embodiment of the first aspect of the invention, the method may also comprise a step of performing a LAMP amplification, or a plurality of LAMP amplifications each comprising the sample (for example provided and/or prepared as described in the “Sample” section herein above) thereby amplifying the target polynucleotide sequence. Each LAMP amplification comprises at least four primers each set flanking the target sequence and LAMP reagents. The LAMP amplification may be performed under isothermal conditions.

The entire LAMP amplification may be prepared in a number of different manners. Any LAMP amplification known to the skilled person may be used with the invention.

One none limiting example LAMP amplification is described here: the FIP anneals to the F2c region on the antisense nucleotide strand of the target DNA. DNA synthesis is initiated by the polymerase, which displaces and releases the DNA strands. Thus, the polymerase synthesizes a strand complementary to the target DNA sequence. Next, the F3 anneals to the F3c region on this antisense strand and initiates a new round of DNA synthesis by the polymerase. The F3-mediated displacement of the antisense strand results in the formation of a stem-loop structure connected by the complementary F1c and F1 regions. Next, the BIP anneals to the DNA strand and complementary DNA is synthesized by the polymerase. Thus, the DNA transforms from a loop to a double stranded linear structure. The DNA strands separate and a dumbbell-like structure with two stem-loops, one at each end of the strand, is formed. This structure serves as the starting point for the amplification cycle. Next, the structure is converted into a stem-loop via self-primed DNA synthesis. The FIP anneals to the single stranded region in the stem-loop and initiates synthesis. In doing so, it releases the complementary strand. The released strand forms a new dumbbell-like structure due to the complementarity between the B1c-B1 and F1-F1c regions, respectively. Starting from the 3′ end of the B1 region, DNA synthesis is initiated. This releases the FIP-linked complementary strand, which in turn forms yet another double-stem-loop structure due to complementarity between the F1-F1c and the B1c-B1 regions, respectively. Next, self-primed DNA synthesis starts over from the 3′ end of the B1 region. The process produces amplification structures consisting of alternately inverted repeats of the target sequence.

The sample may be mixed with the primers and LAMP reagents prior to applying the sample to the electrochemical device.

In contrast to conventional PCR amplification, LAMP amplification is performed using isothermal conditions. In one embodiment, the LAMP amplification can be performed at a constant temperature at 59-75° C., such as 62-73° C., such as 64-70° C., such as 66-68° C.

LAMP Reagents

The method of both the first and second aspects of the invention encompasses performing several LAMP amplifications. The LAMP amplifications will in general comprise a sample, at least four primers flanking the target sequence and LAMP reagents. Said LAMP reagents may be any of the LAMP reagents described herein in this section.

The LAMP reagents, in general, comprise at least nucleotides and a nucleic acid polymerase. The nucleotides may be deoxy-ribonucleotide triphosphate molecules, and preferably the LAMP reagents comprise at least dATP, dCTP, dGTP and dTTP. In some cases, the LAMP reagents also comprise dUTP. The LAMP reagents also comprise a signaling substance, such as H⁺-ions and/or fluorescent dyes.

The nucleic acid polymerase may be any enzyme capable of catalysing template-dependent polymerisation of nucleotides, i.e. replication. The nucleic acid polymerase should tolerate the temperatures used for the LAMP amplification, and it should have catalytic activity at the elongation temperature. Several thermostable nucleic acid polymerases are known to the skilled person.

In some embodiments of both aspects of the invention, the nucleic acid polymerase has high strand displacement activity in addition to a replication activity.

The nucleic acid polymerase may be a bacterial or archaebacterial polymerase. In specific, the nucleic acid polymerase may be Escherichia coli DNA polymerase I. The nucleic acid polymerase may also be Taq DNA polymerase, which has a DNA synthesis-dependent strand replacing 5′-3′ exonuclease activity. Other polymerases include but are not limited to Taq, Tfi, Tzi, Tth, Pwo, Pfu, Q5®, Phusion®, One Taq®, Vent®, Deep Vent®, Klenow (exo-), Bst 2.0 and Bst 3.0 (New England Biolabs, Ipswich, Mass.), PyroPhage® (Lucigen, Middleton, Ws.) Tin DNA polymerase, GspSSD LF DNA polymerase, Rsp (OptiGene, Horsham, UK) and phi29 polymerase.

The Taq DNA polymerase, e.g. obtained from New England Biolabs, can include Crimon LongAmp® Taq DNA polymerase, Crimson Taq DNA Polymerase, Hemo KlenTaq™, or LongAmp® Taq.

The LAMP reagents may comprise a reverse transcriptase (RT). RT is an enzyme capable of generating complementary DNA (cDNA) from an RNA template. Thus, enabling the measurement of RNA. In one embodiment, the LAMP reagents comprise a reverse transcriptase.

In addition, the LAMP reagents may comprise salts, buffers and detection means. The buffer may be any useful buffer, e.g. TRIS. The salt may be any useful salt, e.g. potassium chloride, magnesium chloride or magnesium acetate or magnesium sulfate.

The LAMP reagents may comprise a non-specific blocking agent, such as BSA, gelatin from bovine skin, beta-lactoglobulin, casein, dry milk, salmon sperm DNA or other common blocking agents.

The LAMP reagents may also comprise bio-preservatives (e.g. NaN₃), LAMP enhancers (e.g. betaine, trehalose, etc.) and inhibitors (e.g. RNase inhibitors). Other additives can include dimethyl sulfoxide (DMSO), glycerol, betaine (mono)-hydrate, trehalose, 7-deaza-2′-deoxyguanosine triphosphate (7-deaza-2′-dGTP), bovine serum albumin (BSA), formamide (methanamide), tetramethylammonium chloride (TMAC), other tetraalkylammonium derivatives [e.g. tetraethylammonium chloride (TEA-Cl)]; tetrapropylammonium chloride (TPrA-Cl) or non-ionic detergent, e.g. Triton X-100, Tween 20, Nonidet P-40 (NP-40) or PREXCEL-Q.

Furthermore, the LAMP reagents may also comprise one or more additional means for detection of LAMP amplification product(s). Said means may be any detectable means, and they may be added as individual compounds or be associated with, or even covalently linked to, one of the primers. Detectable means include, but are not limited to, dyes, radioactive compounds, bioluminescent and fluorescent compounds. In a preferred embodiment, the means for detection is one or more probes.

In one embodiment, the LAMP amplification is performed using WarmStart® LAMP Kit (DNA & RNA). This kit contains a Bst 2.0 DNA polymerase, a reverse transcriptase, a nucleotide mix, a visible pH indicator, and a low-buffer solution. The kit furthermore comprises a fluorescent dye.

In another example, the LAMP amplification is performed using WarmStart® Colorimetric LAMP 2× Master Mix (DNA & RNA). This kit contains a Bst 2.0 DNA polymerase, a reverse transcriptase, a nucleotide mix and a buffer solution. The kit furthermore comprises a fluorescent dye.

Primers Flanking the Target Sequence

The method of the second aspect of the invention involves the use of at least four primers that flank the target sequence.

In an embodiment of the first aspect of the invention, the method also involves the use of at least four primers that flank the target sequence.

The four primers may comprise
a forward inner primer also referred to as FIP
a backward inner primer also referred to as BIP
a forward outer primer also referred to as F3
a backward outer primer also referred to as B3

In an embodiment, the FIP has a sequence of SEQ ID NO:3.

In an embodiment, the BIP has a sequence of SEQ ID NO:4.

In an embodiment, the F3 has a sequence of SEQ ID NO:5.

In an embodiment, the B3 has a sequence of SEQ ID NO:6.

The four primers anneal to different parts of the sense and antisense stand of the target polypeptide. See the definitions of the primers in the section “Definitions” as described herein.

The primers are capable of amplifying the target polynucleotide when added to the LAMP reagents under conditions allowing amplification of said target polynucleotide.

In a preferred embodiment, the at least four primers consists of BIP, FIP, F3, and B3.

In one embodiment, the LAMP amplification is performed with an additional pair of loop primers. In one embodiment, the LAMP amplification is performed with a FL primer and a BL primer.

In an embodiment, the FL primer has a sequence of SEQ ID NO:7.

In an embodiment, the BL primer has a sequence of SEQ ID NO:8.

In one embodiment of both aspects of the invention, the LAMP amplification is performed with at least 5 primers, such as at least 6 primers, such as at least 7 primers.

In one embodiment, the LAMP amplification is performed with BIP, FIP, F3, B3, FL and BL primers.

The length of the BIP, FIP, F3, B3, FL and BL primers can depend on the sequence of the target polynucleotide. For example, the length of the primers can be adjusted to achieve a desirable activity at a specific temperature, such as in the range of 59-75° C. Thus, the length of the primers can, individually, be in the range of 10 to 100 nucleotides, for example in the range of 10 to 50 nucleotides, such as in the range of 15 to 20 nucleotides, such as in the range of 15 to 25 nucleotides, such as in the range of 15 to 30 nucleotides, such as in the range of 15 to 40 nucleotides, such as in the range of 15 to 45 nucleotides, such as in the range of 15 to 50 nucleotides in length. Tm of the primers are typically adjusted to in the range of 59 to 75° C.

Primer concentration within the aqueous phase of the LAMP amplifications can, for example, be in the range of 0.05 to 4.0 μM, such as in the range of 0.1 to 3.0 μM, such as in the range of 0.2 to 2.0 μM. One none limiting example hereof is 1.6 μM FIP, 1.6 μM BIP, 0.2 μM F3, 0.2 μM B3, 0.4 μM FL, 0.4 μM BL.

The primers in general, comprise—or even consist—of oligonucleotides. However, in some cases, the primers may comprise nucleotide analogues. Numerous nucleotide analogues are known to the skilled person and include derivatives, wherein a sugar is modified, as in 2′-O-methyl, 2′-deoxy-2′-fluoro, and 2′,3′-dideoxynucleoside derivatives, nucleic acid analogs based on other sugar backbones, such as threose, locked nucleic acids (LNA), LNA derivatives, peptide nucleic acids (PNA), glycol nucleic acid (GNA), threose nucleic acid (TNA), bicyclo sugars, or hexose, glycerol and glycol sugars, nucleic acid analogs based on non-ionic backbones, or nucleic acids and their analogs in non-linear topologies, such as dendrimers, comb-structures, and nanostructures.

The primers may also be linked to various tags (e.g. fluorescent tags, functionalized tags or binding tags), which can optionally be bound to their ends, sugars, or nucleobases.

Primers can be prepared by a variety of methods, including—but not limited to—cloning of appropriate sequences and direct chemical synthesis using methods well known in the art [Narang et al., Methods Enzymol. 68:90 (1979); Brown et al., Methods Enzymol. 68:109 (1979)]. Primers can also be obtained from commercial sources.

In one embodiment, the primers can be designed in manner wherein the primers specifically are capable of amplification of the target polynucleotide sequence.

Measuring an Electrochemical Property of the Solution

The method of the second aspect of the invention comprises a step of measuring, a change in the current and/or potential of the electrochemical cell.

As outlined above, in an embodiment the method of the first aspect of the invention comprises a step of measuring an electrochemical response of the electrochemical cell, such as a change in the current and/or potential and/or impedance of the electrochemical cell.

Said change is preferably a result of a change in a concentration of a signaling substance from a nucleic acid amplification process, for example H⁺, for example a LAMP amplification product or by-product (e.g., H⁺), or a change in a physical quantity as a result of said LAMP amplification. In particular, the change in H⁺ results in a change in the oxidation state a quinone, a quinone derivative, and/or a pH indicator which is dissolved in the electrochemical cell, which is measured as change in electrochemical response of the electrochemical cell. Measurement of the change in the electrochemical response (e.g., current and/or potential and/or impedance) of the electrochemical cell of the electrochemical device preferably takes place following contacting the electrochemical device/two or three-electrode electrochemical cell with a measurement setup, for forming an electrochemical system.

The electrochemical system comprises typically the electrochemical device/two or three-electrode electrochemical cell, including, where present, the film electrodes, the solution and a separate current path, typically a potentiostat. The electrochemical system thereby comprises multiple film electrodes, such as a working electrode, a counter electrode and a reference electrode, spatially separated and distributed throughout one or more ionically-conductive media, or electrolytes, while also being in electrical contact with one another via a separate current path (preferably comprising, or consisting of, a potentiostat).

The electrodes in an electrochemical system undergo oxidation and reduction reactions, with movement of electrons producing current traveling through the current path simultaneously with movement of ions through the media producing an overall balance of charge transfer within the system.

A potentiostat is typically an instrument designed to control the electrodes in an electrochemical system by adjusting the potential (or current) and measuring the subsequent effect on current (or potential). This task is typically accomplished through a variety of internal circuits, operational amplifiers, and feedback loops. External cables are commonly used to physically connect to each electrode, which typically includes a working electrode, a counter electrode, and a reference electrode.

The measurements may be performed according to any electroanalytical method, for example open circuit potential, potentiometric, impedimetric, coulometric, voltametric and/or amperometric measurements. Typically a measurement of a solution may take between 1 and 90 minutes, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 minutes.

In one embodiment, the measurement of a solution may take within the range of 1 to 45 minutes, such as 1 to 30 minutes such as 1 to 15 minutes, such as 5 to 10 minutes.

Voltammetry is the study of current as a function of applied potential. Thereby, voltammograms I=f(E) curves are produced. Typically in such a measurement, the potential is varied arbitrarily either step by step or continuously, and the actual current value is measured as the dependent variable. The opposite is amperometry. The shape of the curves depends on the speed of potential variation (nature of driving force) and on whether the solution is stirred or quiescent (mass transfer).

For quantification, analysis requires the consideration of kinetics in addition to thermodynamics, due to the temporal component of voltammetry. Idealized theoretical electrochemical thermodynamic relationships such as the Nernst equation are modeled without a time component. While these models are insufficient alone to describe the dynamic aspects of voltammetry, models like the Tafel equation and Butler-Volmer equation lay the groundwork for the modified voltammetry relationships that relate theory to observed results.

### Specific Embodiments of the First Aspect of the Invention

Specific embodiments of the first aspect of the invention are provided in the numbered paragraphs below.

X1. A method of measuring the pH of a solution, wherein the method comprises the steps of:
providing a solution comprising a compound capable of undergoing a change in its oxidation state and/or structural conformation as a function of the pH of the solution;
applying the solution to a two or three-electrode electrochemical cell;
measuring an electrochemical response of the electrochemical cell; and
quantifying the pH of the solution as a function of the electrochemical response of the electrochemical cell.

X2. The method according to paragraph X1, wherein the compound capable of undergoing a change in its oxidation state and/or structural conformation as a function of the pH of the solution is a catechol, a catechol derivative and/or a compound comprising a catecholic group.

X3. The method according to any preceding paragraph, wherein the compound capable of undergoing a change in its oxidation state and/or structural conformation as a function of the pH of the solution is a quinone a quinone derivative, or a pH indicator, or combinations thereof.

X4. The method according to any preceding paragraph, wherein the compound is a quinone or quinone derivative selected from the list consisting of 1,2-benzoquinone, 1,4-benzoquinone, 1,4-naphthoquinone, 9,10-anthraquinone, and derivatives and combinations thereof.

X5. The method according to any preceding paragraph, wherein the compound capable of undergoing a change in its oxidation state and/or structural conformation as a function of the pH of the solution is a pH indicator selected from the list consisting of malachite green oxalate, brilliant green, eosin yellowish, erythrosine B, methyl green, methyl violet, picric acid, cresol red, crystal violet, m-Cresol purple, thymol blue, p-Xylenol blue, Eosin (bluish), quinaldine red, 2,4-dinitro phenol, 4-(dimethylamino) azobenzol, bromochlorophenol blue, bromophenol blue, congo red, methyl orange, bromocresol green, 2,5-dinitrophenol, alizarin sulphonic acid, methyl red, chlorophenol red, litmus, bromocresol purple, bromophenol red, 4-nitrophenol, bromoxylenol blue, bromothymol blue, phenol red, 3-nitrophenol, neutral red, creosol red, 1-naphtholphthalein, m-cresol purple, thymol blue, p-xylenol blue, phenolphthalein, thymolphthalein, alkali blue, alizarin yellow GG, indigo carmine, epsilon blue, titan yellow, and combinations thereof.

X6. The method according to any preceding paragraph, wherein the compound capable of undergoing a change in its oxidation state and/or structural conformation as a function of the pH of the solution is dissolved in the solution.

X7. The method according to any preceding paragraph, the electrochemical response of the electrochemical cell that is measured is the potential of the electrochemical cell, the current of the electrochemical cell, the impedance of the electrochemical cell, or a combination of these.

X8. The method according to paragraph X6, wherein the potential of the electrochemical cell is measured via cyclic squarewave voltammetry, squarewave voltammetry, linear sweep voltammetry, cyclic voltammetry or open circuit potentiometry.

X9. The method according to any preceding paragraph, wherein the concentration of the compound capable of undergoing a change in its oxidation state and/or structural conformation as a function of the pH of the solution within the solution is from about 1 μM to about 1000 μM, such as from about 1 μM to about 500 μM, for example from about 5 μM to about 250 μM, in particular from about 50 μM to about 200 μM.

X10. The method according to any preceding paragraph, wherein the step of quantifying the pH of the solution as a function of the electrochemical response of the electrochemical cell is performed via application of a regression algorithm.

X11. The method according to any preceding paragraph, wherein the solution comprises a buffer.

X12. The method according to any preceding paragraph, wherein the solution comprises a sample, optionally wherein the sample comprises human, animal plant, bacterial, fungal, or protozoan cells.

X13. The method according to any preceding paragraph, wherein the solution further comprises a sample, preferably wherein the sample is selected from the list consisting of a nasal sample, a throat sample, an anal sample, a vaginal sample, an ear draining sample, a skin surface swab sample, a urine sample, a whole blood sample, a serum sample, a plasma sample and a lymph drainage sample.

X14. The method according to any preceding paragraph, wherein the solution further comprises primers and nucleic acid amplification reagents, preferably wherein the nucleic acid amplification reagents are LAMP reagents.

X15. The method according to paragraph X13, wherein the nucleic acid amplification reagents comprise a signaling substance, or are capable of releasing a signaling substance, or comprise both a signaling substance and a substance capable of release a signaling substance.

X16. The method according to paragraph X14, wherein the signaling substance is H.

X17. The method according to any one of paragraphs X13 to X15, wherein the method comprises a step of performing a nucleic acid amplification step, preferably a plurality of nucleic acid amplification steps.

X18. The method according to paragraph X16, wherein the nucleic acid amplification step(s) is/are a loop-mediated isothermal amplification (LAMP) step.

X19. The method according to any one of paragraphs X13 to X17, wherein the method is for determining the presence of a target polynucleotide in the solution.

X20. The method according to paragraph X18, wherein the step of measuring the electrochemical response of the electrochemical cell comprises measuring a change in the current and/or potential of the electrochemical cell due to a concentration change of the signaling substance.

X21. The method according to any one of paragraphs X13 to X19, wherein the LAMP reagents comprise a reverse transcriptase.

X22. The method according to any one of paragraphs X13 to X20, wherein the primers comprise a forward inner primer, a backward inner primer, a forward outer primer and a backward outer primer.

X23. The method according to paragraph X21, wherein the LAMP amplification is performed with an additional pair of loop primers, such as forward loop primer and/or backward loop primer.

X24. The method according to any of the preceding paragraphs, wherein the three-electrode electrochemical cell comprises a working electrode, a reference electrode and a counter electrode.

X25. The method according to paragraph X23, wherein the working electrode comprises an ion selective membrane, located such that it excludes selected ions of the solution from contacting the working electrode.

X26. The method according to paragraph X23 or paragraph X24, wherein the surface of the working electrode is chemically modified to allow for binding of OH and H₃O⁺ ions or is reactive to the pH of the solution.

X27. The method according to any one of paragraphs X23 to X25, wherein the material of the reference electrode and the counter electrode is Ag/AgCl.

X28. The method according to any one of paragraphs X23 to X26, wherein the material of the working electrode is carbon.

X29. The method according to any of the preceding paragraphs, wherein the electrodes comprise or consist of gold, silver, carbon, platinum, ruthenium dioxide, or a combination thereof.

X30. The method according to any of the preceding paragraphs, wherein the material of the electrodes are identical or different.

X31. The method according to any of the preceding paragraphs, wherein the electrodes of the two or three-electrode electrochemical cell are film electrodes, preferably screen printed electrodes.

X32. The method according to any of the preceding paragraphs, wherein the electrodes of the two or three-electrode electrochemical cell are wire electrodes.

X33. The method according to any of the preceding paragraphs, wherein the two or three-electrode electrochemical cell comprises a substrate onto which the electrodes are located, and wherein the material of said substrate is a chemically inert material, such as alumina, ceramic, glass, or an inert plastic.

X34. The method according to any of the preceding paragraphs, wherein the two or three-electrode electrochemical cell comprises a microfluidic inlet, in fluidic connection with the electrochemical cell, for receiving the solution.

X35. The method according to any of the preceding paragraphs, wherein, following application of the solution, a vapour barrier is added for preventing evaporation of the solution.

X36. The method according to paragraph X33, wherein the vapor barrier is formed by fluidly sealing the inlet of the three-electrode electrochemical cell, or the microwell, with a physical lid, a heated lid, a mineral oil, and/or a paraffin wax.

X37. The method according to any of the preceding paragraphs, wherein a hydrophilic area connects the microfluidic inlet and the two or three-electrode electrochemical cell, such as for capillary filling of the two or three-electrode electrochemical cell.

X38. The method according to any of the preceding paragraphs, wherein the measurements are performed by using a potentiostat or similar circuit.

X39. The method according to any of the preceding paragraphs, wherein the measurements are performed for a period of 1 minute to 90 minutes.

X40. The method according to any one of the preceding paragraphs, wherein the nucleotide amplification is performed at a constant temperature in the range of from 59 to 75° C., such as from 62 to 73° C., such as from 64 to 70° C., such as from 66 to 68° C.

Y. Use of a two or three-electrode electrochemical cell for measuring the pH of a solution, said use comprising:
providing a solution comprising a compound capable of undergoing a change in its oxidation state and/or structural conformation as a function of the pH of the solution;
applying the solution to the two or three-electrode electrochemical cell;
measuring an electrochemical response of the electrochemical cell; and
quantifying the pH of the solution as a function of the electrochemical response of the electrochemical cell.

Y1. Use of a three-electrode electrochemical cell for measuring the pH of a solution, said use comprising:
providing a solution comprising a compound capable of undergoing a change in its oxidation state and/or structural conformation as a function of the pH of the solution;
applying the solution to the three-electrode electrochemical cell;
measuring an electrochemical response of the electrochemical cell; and
quantifying the pH of the solution as a function of the electrochemical response of the electrochemical cell.
Y2. A system for measuring the pH of a solution, the system comprising:
   b. An instrument comprising a potentiostat; and
      v. A first receptacle comprising a three-electrode electrochemical cell, and a second receptacle comprising a quinone, a quinone derivative, and/or a pH indicator, or
      vi. A first receptacle comprising a three-electrode electrochemical cell and a quinone, a quinone derivative, and/or a pH indicator,
   wherein the potentiostat is configured to measure an electrochemical response of the electrochemical cell.

Y3. The system according to claim 39, wherein the quinone, quinone derivative, and/or pH indicator are dissolved in an aqueous solution in the first or in the second receptacle.

Y4. The system according to any one of the preceding paragraphs, wherein the potentiostat is configured to measure an electrochemical response of the electrochemical cell.

Y5. The system according to any one of the preceding paragraphs, wherein the potentiostat is configured to measure an electrochemical response, wherein the electrochemical response is representative of the oxidation state of the quinone, quinone derivative, and/or pH indicator in the first receptacle.

Y6. The system according to any one of the preceding paragraphs, wherein the system further comprises a heating unit, configured for heating the first and/or second receptacle, such as to a temperature within the in the range of from 59° C. to 75° C.

Y7. The system according to any one of the preceding paragraphs, wherein the first receptacle accommodates a nucleic acid amplification reaction system, such as at least four primers configured to flank a target polynucleotide sequence and LAMP reagents.

Y8. The system according to any one of the preceding paragraphs, wherein the first receptacle comprises a microfluidic inlet for receiving a solution and/or a sample.

Y9. The system according to claim 44, wherein the microfluidic inlet is connected to the electrochemical cell by a hydrophilic zone, such as a porous/fibrous structure or a hydrophilic channel, configured to transport the sample by capillary action.

Y10. The system according to any one of the preceding paragraphs, wherein the first receptacle and/or the second receptacle is an Eppendorf tube, a microwell, or a culture flask.

Y11. The system according to any one of the preceding paragraphs, wherein the electrodes are as defined herein.

A particular method of quantifying a target polynucleotide in a sample using LAMP In a second aspect of the invention there is provided a method of quantifying a target polynucleotide in a sample, said method comprising the steps of:
a. Providing a sample;
b. Applying the sample to an electrochemical device comprising an electrochemical cell having multiple film electrodes;
c. Performing, if the target polynucleotide is present in the sample, a plurality of isothermal LAMP amplifications, each comprising the sample, at least four primers each flanking the target polynucleotide and LAMP reagents comprising a signaling substance and/or capable of releasing a signaling substance, wherein each LAMP amplification releases or consumes the signaling substance;
d. Measuring, simultaneously as step e., a change in the current and/or potential of the electrochemical cell due to a concentration change of the signaling substance; and
e. Deriving an amplification rate, by the rate of the change of the current and/or potential, thereby quantifying the target polynucleotide in the sample.

For the avoidance of doubt, any aspects of the second aspect of the invention may be combined with any aspects of the first aspect of the invention as detailed in any of the sections above.

In an embodiment of the second aspect of the invention the sample is mixed with the primers and LAMP reagents prior to applying the sample to the electrochemical device.

In another embodiment of the second aspect of the invention the electrochemical device comprises the primers and the LAMP reagents.

In a further embodiment of the second aspect of the invention the multiple film electrodes comprise a working electrode, a reference electrode and a counter electrode.

In an embodiment of the second aspect of the invention the working electrode comprises an ion selective membrane, located such that it excludes selected ions of the solution from contacting the working electrode.

In another embodiment of the second aspect of the invention the surface of the working electrode is chemically modified to allow for binding of OH⁻ and H₃O⁺ ions or is reactive to the pH of the solution.

In an embodiment of the second aspect of the invention the surface of any of the electrodes comprises, or is chemically modified by, ruthenium oxide, graphene platelets, or chemically modified such that the electrodes have exposed thiol/hydroxyl groups.

In another embodiment of the second aspect of the invention the material of the reference electrode and the counter electrode is Ag/AgCl at around 60/40 w/w ratio.

In a further embodiment of the second aspect of the invention the material of the working electrode is carbon.

In an embodiment of the second aspect of the invention the electrodes comprise or consist of gold, silver, carbon, platinum, ruthenium dioxide, or a combination thereof.

In another embodiment of the second aspect of the invention the material of the electrodes are identical or different.

In a further embodiment of the second aspect of the invention the film electrodes are screen printed electrodes.

In a further embodiment of the second aspect of the invention the film electrodes are wire electrodes.

In an embodiment of the second aspect of the invention the electrochemical device comprises a substrate onto which the electrodes are located, and wherein the material of said substrate is a chemically inert material, such as alumina, ceramic, glass, or an inert plastic.

In another embodiment of the second aspect of the invention the electrochemical device comprise a microfluidic inlet, in fluidic connection with the electrochemical cell, for receiving the sample.

In a further embodiment of the second aspect of the invention following application of the solution, a vapour barrier is added for preventing evaporation of the solution.

In an embodiment of the second aspect of the invention the vapor barrier is formed by fluidly sealing the inlet of the electrochemical device, or the microwell, with a physical lid, a heated lid, a mineral oil, and/or a paraffin wax.

In another embodiment of the second aspect of the invention a hydrophilic area connects the microfluidic inlet and the electrochemical cell, such as for capillary filling of the electrochemical device.

In a further embodiment of the second aspect of the invention the measurements are performed by using a potentiostat or similar circuit.

In an embodiment of the second aspect of the invention the measurements comprise or consist of, potentiometric, impedimetric, coulometric, voltammetric and/or amperometric measurements.

In another embodiment of the second aspect of the invention the solution is measured between 1 and 90 minutes.

In a further embodiment of the second aspect of the invention the LAMP reagents comprise a reverse transcriptase.

In an embodiment of the second aspect of the invention the nucleotide amplification is performed at a constant temperature in the range of 59-75° C., such as 62-73° C., such as 64-70° C., such as 66-68° C. ° C.

In another embodiment of the second aspect of the invention the primers comprise a forward inner primer, a backward inner primer, a forward outer primer and a backward outer primer.

In a further embodiment of the second aspect of the invention the LAMP amplification is performed with an additional pair of loop primers, such as forward loop primer and/or backward loop primer.

In another embodiment, the present invention relates to a method of determining the presence of a target polynucleotide in a sample. The method comprises discrimination between a target polynucleotide positive sample and a target polynucleotide negative sample by quantifying and/or determining the polynucleotide amplification rate.

Sequences

SEQ ID NO: 1
ORF1 fragment of SARS-CoV-2
CCCTATGTGTTCATCAAACGTTCGGATGCTCGAACTGCACCTCATGGTCA
TGTTATGGTTGAGCTGGTAGCAGAACTCGAAGGCATTCAGTACGGTCGTA
GTGGTGAGACACTTGGTGTCCTTGTCCCTCATGTGGGCGAAATACCAGTG
GCTTACCGCAAGGTTCTTCTTCGTAAGAACGGTAATAAAGGAGCTGGTGG
CCATAGTTACGGCGCCGATCTAAAGTCATTTGACTTAGGCGACGAGCTTG
GCACTGATCCTTATGAAGA
SEQ ID NO: 2
Gene N fragment of SARS-CoV-2
ATGACCAAATTGGCTACTACCGAAGAGCTACCAGACGAATTCGTGGTGGT
GACGGTAAAATGAAAGATCTCAGTCCAAGATGGTATTTCTACTACCTAGG
AACTGGGCCAGAAGCTGGACTTCCCTATGGTGCTAACAAAGACGGCATCA
TATGGGTTGCAACTGAGGGAGCCTTGAATACACCAAAAGATCACATTGGC
ACCCGCAATCCTGCTAACAATGCTGCAATCGTGCTAC
SEQ ID NO: 3
Forward Inner Primer (FIP)
AGGTGAGGGTTTTCTACATCACTATATTGGAACAAGCAAATTCTATGG
SEQ ID NO: 4
Backward Inner Primer (BIP)
ATGGGTTGGGATTATCCTAAATGTGTGCGAGCAAGAACAAGTG
SEQ ID NO: 5
Forward Outer Primer (F3)
CCACTAGAGGAGCTACTGTA
SEQ ID NO: 6
Backward Outer Primer (B3)
TGACAAGCTACAACACGT
SEQ ID NO: 7
Forward Loop Primer (FL)
CAGTTTTTAACATGTTGTGCCAACC
SEQ ID NO: 8
Backward Loop Primer (BL)
TAGAGCCATGCCTAACATGCT

### EXAMPLES

### Example 1

### Quantitative Electroanalytical Measurements of a Nasal Swab Sample by LAMP Amplification

### Materials and Methods

### Screen Printed Electrode

i. A screen printed pH sensitive 3-electrode system is used, the system comprising 3 screen printed electrodes which serve as working electrode, reference electrode and auxiliary electrode. The system is printed on chemically inert alumina.
   1. The material of the reference electrode is Ag/AgCl (60/40 ratio), with an additional layer providing a fixed concentration of chloride ions for a true reference electrode measurement.
   2. The material of the working electrode is Carbon, with pH sensing layer of a solid state membrane with combination of Cobalt Oxide and Iridium Oxide.
   3. The material of the counter electrode is Ag/AgCl electrode (60/40 ratio), with an additional layer providing a fixed concentration of chloride ions for a true reference electrode measurement.
ii. Above the electrodes is superimposed a microfluidic sample port in the form of a microwell
iii. A vapor barrier for the microwell is used, in the form of a physical lid.

Primers

A 10× Primer Mix containing 16 μM FIP, 16 μM BIP, 2 μM F3, 2 μM B3, 4 μM LoopF, 4 μM LoopB in nuclease free water (not TE). Final concentration in the amplification reaction mix before the reaction commences are 1.6 μM FIP, 1.6 μM BIP, 0.2 μM F3, 0.2 μM B3, 0.4 μM LoopF, 0.4 μM LoopB.

Sample
i. A nasal swab sample inoculated in the Viral Transport Medium is used after heat treatment at 80° C. for 1 minute. An example of the Viral Transport Medium used is Sterile Hanks Balanced Salt Solution (HBSS) 1× with calcium and magnesium ions, with or without Phenol Red, 2% v/v Fetal Bovine Serum, 100 μg/mL Gentamicin sulfate and 0.5 μg/mL for Amphotericin B.

Amplification Reaction
i. 2 uL sample is mixed with
   a. 2.5 uL of 25×LAMP Primer Mix targeting the genomic area of interest (final concentration of 1.6 uM FIP, 1.6 uM BIP, 0.2 uM F3, 0.2 uM B3, 0.4 uM LOOP F, 0.4 uM LOOP B),
   b. 12.5 uL of WarmStart Colorimetric LAMP 2× Master Mix from New England Biolabs Inc., and
   c. Nuclease free water to a final volume of 25 uL.

Amplification Conditions and Assay Design
1. The amplification reaction mixture (with the sample) is added immediately after mixing on the pH sensor pre-heated at 65° C., already connected to a potentiostat.
2. The vapor barrier is put into place to avoid evaporation.
3. Potentiometric measurements are run realtime and the voltage is measured continuously for 30 minutes.

Interpretation
i. The presence of the nucleic acid template is indicated during the 30 minutes by a drop in pH (pH sensing). The time differential of the change is correlated with quantity of the nucleic acid template.

### Example 2

### Continuous Electrochemical Real Time Quantitative Monitoring of Loop Mediated Isothermal Amplification in Low Tris Buffer Using Screen Printed Electrodes.

### Materials and Methods

### Screen Printed Electrode

i. A screen printed pH sensitive 3-electrode system comprising of 3 screen printed electrodes which serve as working electrode, reference electrode and auxiliary electrode, printed on a chemically inert substrate like alumina or ceramic or glass or inert plastic substrates.
ii. The electrodes may be made of same or different materials like gold, silver, carbon, platinum, ruthenium dioxide, etc.
iii. The working electrode may incorporate a layer of ion selective membrane, and/or the electrode surface may be modified to allow binding of OH⁻ and H3O⁺ ions or is sensitive to pH of the solution when exposed to the solution via direct contact. Such modifications may comprise of Ruthenium oxide, graphene platelets, exposed thiol groups, exposed hydroxyl groups, etc.
iv. Setup:
   1. The reference electrode comprises of a Ag/AgCl electrode (60/40 ratio), with an (optional) additional layer providing a fixed concentration of chloride ions for a true reference electrode measurement.
   2. The Working electrode comprises of Carbon
   3. The counter electrode comprises of a Ag/AgCl electrode (60/40 ratio), with an (optional) additional layer providing a fixed concentration of chloride ions for a true reference electrode measurement.
   v. Above the electrodes is superimposed a microfluidic sample port in the form of a microwell (preferred) or other sample introduction/containment methods.
   vi. A vapor barrier for the microwell may be used. Alternatives may include physical lid, heated lid, mineral oil vapor barriers, paraffin wax vapor barriers.

Primers

A 10× Primer Mix containing 16 μM FIP, 16 μM BIP, 2 μM F3, 2 μM B3, 4 μM LoopF, 4 μM LoopB in nuclease free water (not TE). Final concentration in the amplification reaction mix before the reaction commences are 1.6 μM FIP, 1.6 μM BIP, 0.2 μM F3, 0.2 μM B3, 0.4 μM LoopF, 0.4 μM LoopB

Samples
ii. A nasal or throat swab sample inoculated in the Viral Transport Medium may be used directly or after heat treatment at 70-90° C. for 1 minute.
iii. An anal or vaginal or ear drainage or skin surface swab sample inoculated in the Viral Transport Medium may be used directly or after heat treatment at 70-90° C. for 1 minute.
iv. A urine sample may be used may be used directly or after heat treatment at 70-90° C. for 1 minute.
v. A whole blood sample mixed with an anticoagulant like heparin, EDTA or sodium citrate, with optional additional lysis via mechanical lysis, thermal lysis, acoustic cavitation, osmotic shock, enzymatic lysis or chemical lysis.
vi. A lymph drainage sample or a serum sample may be used directly or after heat treatment at 70-90° C. for 1 minute.
vii. Purified nucleic acid content from a biological sample or a crude nucleic acid extract

Amplification Reaction
i. Typical amplification reactions contain primers (e.g. four, five or six primers), sample (which may or may not contain template to which the primers bind), nucleotides (corresponding to G, A, T and C), a buffering agent (1 mM to 5 mM Tris or 1.5 mM to 5 mM Tris or an equivalent buffer thereof), one or more salts (e. g., (NH) 2SO4, NaCl, MgSO4, MgCl2, etc.), a bacterial or archaebacterial polymerase (which may or may not be thermostable and may or may not have strand displacing activity), and any necessary cofactors and optional detergents, etc. Examples of thermostable polymerases that can be used in a PCR or polymerases for use in isothermal amplification reactions include, but are not limited to Taq, Tfi, Tzi, Tth, Pwo, Pfu, Q5®, Phusion®, One Taq®, Vent®, Deep Vent®, Klenow (exo-), Bst 2.0 and Bst 3.0 (New England Biolabs, Ipswich, Mass.), PyroPhage® (Lucigen, Middleton, Wis.), Tin DNA polymerase, GspSSD LF DNA polymerase, Rsp (OptiGene, Horsham, UK) and phi29 polymerase, etc.
ii. Preferred reaction mixture:
   2 uL sample (containing DNA/RNA or No-template control; range: 1-5 uL; more than 2 uL sample if a Transport medium is being used inhibits reaction; less than 2 uL will mean there might be too little template) is mixed with
   a. 2.5 uL of 25×LAMP Primer Mix targeting the genomic area of interest (final concentration of 1.6 uM FIP, 1.6 uM BIP, 0.2 uM F3, 0.2 uM B3, 0.4 uM LOOP F, 0.4 uM LOOP B),
   b. 12.5 uL of WarmStart Colorimetric LAMP 2× Master Mix from New England Biolabs Inc. (also contains reverse transcriptase), and
   c. Nuclease free water to a final volume of 25 uL.
iii. Alternative reaction mixture:
   2 uL sample (containing DNA/RNA or No-template control; can be up to 5 uL) is mixed with
   a. 2.5 uL of 25×LAMP Primer Mix targeting the genomic area of interest (final concentration of 1.6 uM FIP, 1.6 uM BIP, 0.2 uM F3, 0.2 uM B3, 0.4 uM LOOP F, 0.4 uM LOOP B),
   b. 12.5 uL of WarmStart LAMP 2× Master Mix from New England Biolabs Inc., and
   c. Nuclease free water to a final volume of 25 uL.

Amplification Conditions and Assay Design
i. Preferred
   1. The amplification reaction mixture (with the sample) is added immediately after mixing on the pH sensor pre-heated at 59-75° C., already connected to a potentiostat or equivalent circuit.
   2. The vapor barrier is put into place to avoid evaporation.
   3. Potentiometric measurements are run realtime and the voltage is measured continuously for 1-90 minutes.
   4. The assay can be parallelized for multiple samples.
ii. Alternative
   1. The mixing of the sample with the rest of the components may happen directly on the chip using a pipette or microfluidic mixing.
   2. The amplification reaction mixture (with the sample) is added in a PCR or microfuge tube and heated at 59-75° C. The sample is aliquoted after 1-90 minutes, or at intervals of 1-5 minutes 2-5 times, and the pH is measured on the pH sensor at room temperature already connected to a potentiostat or equivalent circuit.
   3. Instead of pH, impedimetric measurements, coulometric measurements, amperometric measurements may be performed without the use of any additional redox probe or DNA intercalating dye.

Interpretation
i. The presence of the nucleic acid template is indicated between 1-90 minutes by
   i. A drop in pH (pH sensing)
   ii. A drop in conductivity (amperometry)
   iii. Increase in impedance (impedimetry)
   iv. Change in net charge (coulometry)
ii. Further, the time differential of this change is correlated with quantity of the nucleic acid template

### Example 3

### A Three-Electrode System Method for Monitoring the pH of a Solution Containing Phenol Red

### Parameters Used

In this example, 50 μL of an aqueous solution containing PBS, NaOH or HCl (for pH adjust), 0.1 M KCl as supporting electrolyte and 100 uM phenol red as pH probe was analysed.

The aqueous solution was introduced into a three-electrode electrochemical cell and squarewave voltammetry was performed on the electrochemical cell with the following parameters:
a. Peak at 0.5 V vs. Ag/AgCl. Scan range 0 to 0.6 V vs. Ag/AgCl; frequency: 10 Hz; potential step: 5 mV; Amplitute: 10 mV.
b. Peak at between −0.4 to −0.55 V vs. Ag/AgCl. Scan range 0 to −0.7 V vs. Ag/AgCl; frequency: 10 Hz; potential step: 5 mV; Amplitute: 10 mV.

N₂0₃-Au disposable electrodes were used along with an Anapot potentiostat.

The pH change was analysed as a function of peak height (i) and peak position which is defined by the potential (E):

ΔpH=f(i*E)+f(i)+f(E)

Results of Analysis

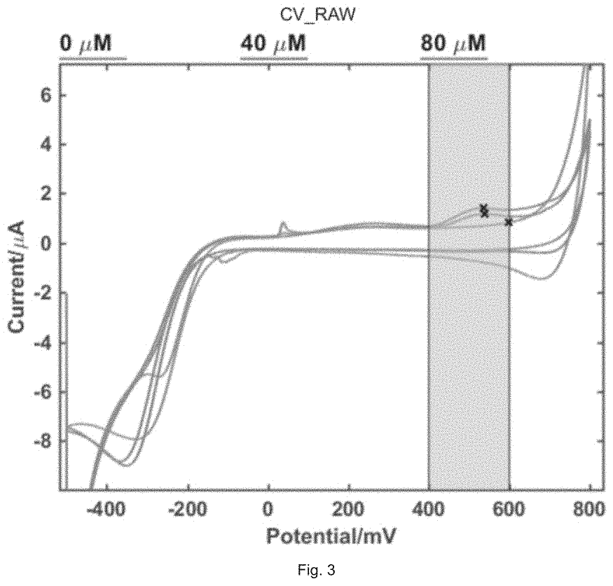

Phenol red is a tri-quinone structured chemical, which undergoes three-electron transfer in the potential window of −1V to 1V (vs Ag/AgCl, see FIG. 3 for an overview of redox peaks for phenol red). The peak current and peak potential are both pH dependent. Despite its limited visible light spectrum between pH 6-pH 8, the redox potentials of phenol red and the corresponding peak currents are capable of the full pH range identification.

Phenol red is the simplest form of the sulfonephthalein group and a weak acid with a pKa of 7.9 and is one of the most commonly used pH indicators. Its chemical structure and the dissociation in aqueous solution can be seen in the following scheme:

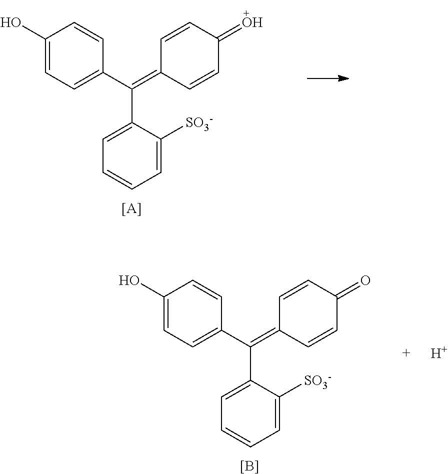

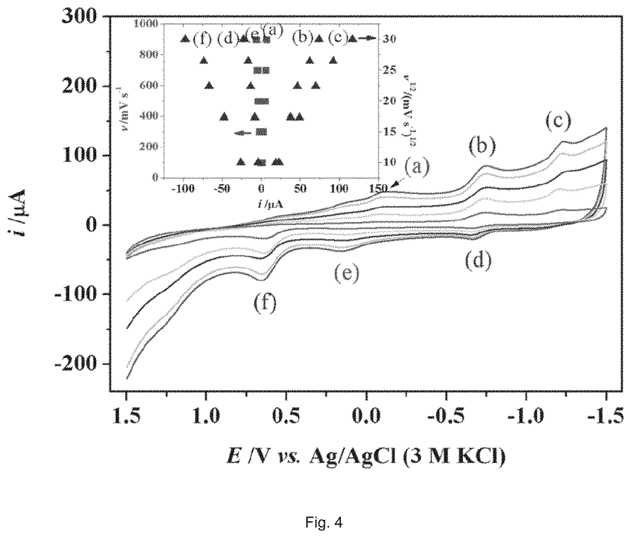

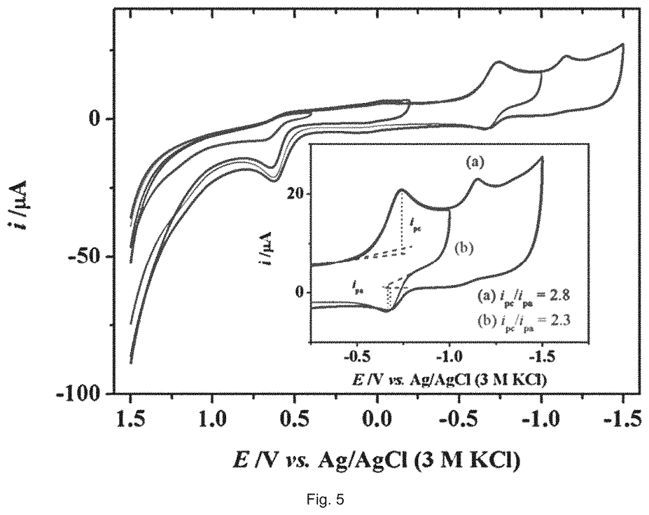

The ionizable sector of quinone methide in the tri-aromatic structure of phenol red imparts rich redox properties which shows advantageous characteristics for various applications. The sector of quinone methide in the structure is the center of the rich redox chemistry. Cyclic voltammograms of phenol red scanned at varying scan rates with a bare GC electrode in solutions buffered at pH 7.9 in PBS are shown in FIG. 4. Six significant waves can be observed in FIG. 4. By correlating peak currents with the values of scan rate and square root of scan rate, as shown in the inset plot of FIG. 4, it was found that the waves (b), (c), (d), and (f) can be categorized as redox reactions of phenol red at the electrode interface, since the fact that peak current showing proportional to square root of scan rate is characteristic of diffusion-controlled reactions. In contrast, the waves (a) and (e) are categorized as adsorption process of phenol red on the electrode surface, due to the linear relation of peak current with the scan rate. In order to identify the redox couples of phenol red, cyclic voltammetry scans of phenol red were conducted at varying switching potentials, the results of which are shown in FIG. 5. The scans were initiated cathodically from 1.5 V to switching potential of 0.4, −0.2, −1.0, and −1.5 V, respectively. Looking to the far right of the curves in the inset graph in FIG. 5, curve (a) shows two reduction peaks with only one significant oxidation peak whereas curve (b) shows simply one redox couple. The ipc/ipa ratio for curve (a) is about 2.8 and that of curve (b) is 2.3, indicating a decline of reduced species in the process of curve (a).

The following two-step reduction may account for the finding:

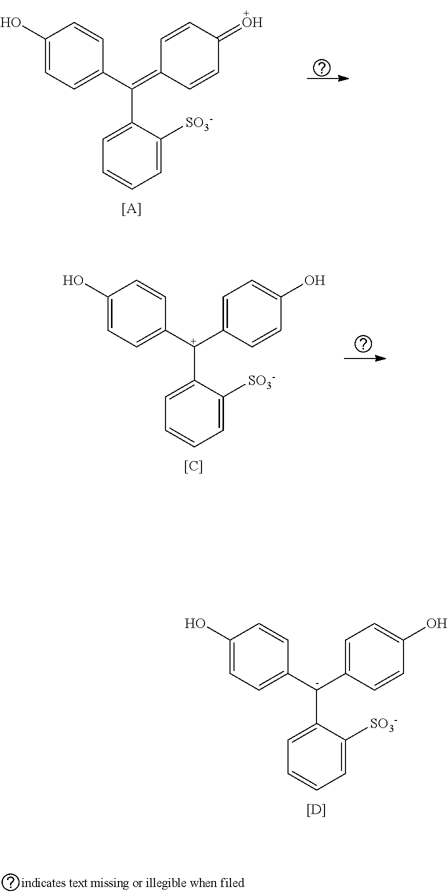

The peak of the first reduction took place at −0.75 V followed by the second reduction situated at −1.12 V in curve (a) of the inset graph in FIG. 5. Moreover, a disproportionation consumes a fraction of the free radical product of the first reduction [C] into a carbanion [D] and the starting oxonium ion [A]:

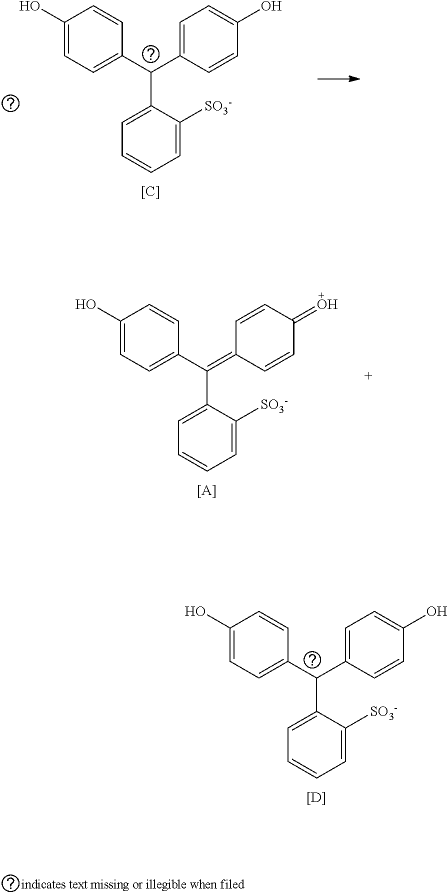

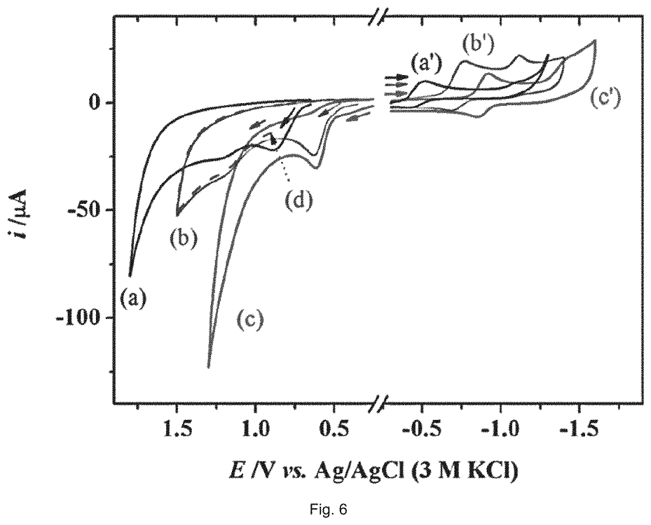

This causes the decline of both oxidation peaks in curves (a) and (b) and hence the deviation of i_{pc}/iₚₐ ratio from a general reversible process. In addition, because the protonation of the carbanion species is an irreversible reaction, the second reduction peak at −1.12 V is much smaller compared with that of the first counterpart at −0.75 V. Based on the aforementioned, it accounts for the relatively lower i_{pc}/iₚₐ in curve (a) compared to that of curve (b). The influence of pH value on the reductions and oxidations of phenol red are illustrated in FIG. 6, solution of pH 3.9 (curves (a) and (a′), [A] being the main species), 7.9 (curves (b) and (b′), equal in amount of both forms), and 11.9 (curves (c) and (c′), mainly [B]) containing phenol red were buffered with PBS and CV scans of these solutions were conducted both in anodic and cathodic directions. It is worth noting that, due to the dependence of peak potential on pH variation, the first reductions of (a′) to (c′) include one electron and one proton transfer while the second reduction proceeds without proton transfer.

The corresponding anodic scans of the solutions (a) to (c) exhibit two oxidation peaks for curves (a) and (b) despite only one for (c). For the cases of (a) and (b), the unprotonated phenol red [B] undergoes first oxidation and followed by another oxidation of the protonated counterpart [A] at a more positive potential accompanied by deprotonation. In order to clarify the connection between both oxidations, a scan started before the potential of second oxidation (0.90 V) was further conducted as shown in curve (d). The result suggests that the second oxidation is not derived from the product of the first oxidation. By contrast, the absence of the second oxidation in curve (c) is owing to a shortfall of [A] in an alkaline environment. Therefore, the oxidations from both species of phenol red result in a radical cation [E], as revealed by the following routes:

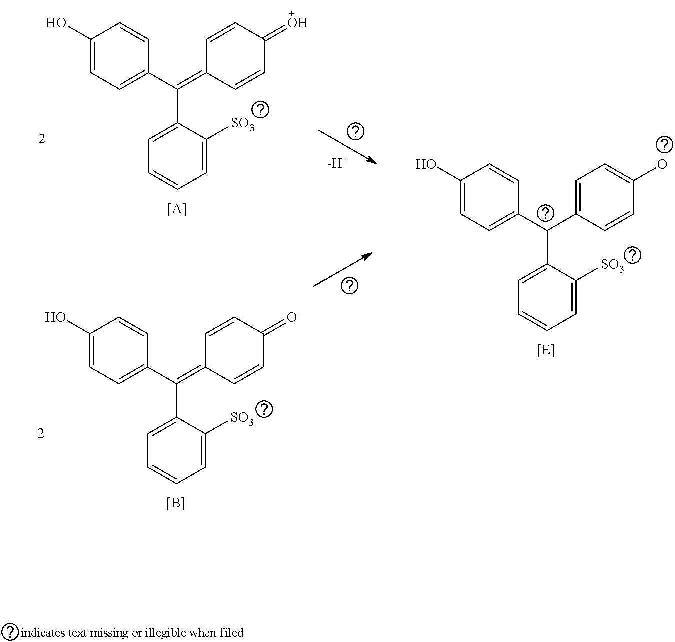

Squarewave voltammetry is used for the signal identification, benefiting from excellent signal enhancement as compared to cyclic voltammetry, facilitating simple signal processing. The optimal concentration of Phenol Red was observed to be around 100 uM. Without wishing to be bound by theory, 100 μM gives well defined redox peak without inhibiting nuclei amplification. However, a wide range of phenol red concentrations may be used with signal identification still being achieved.

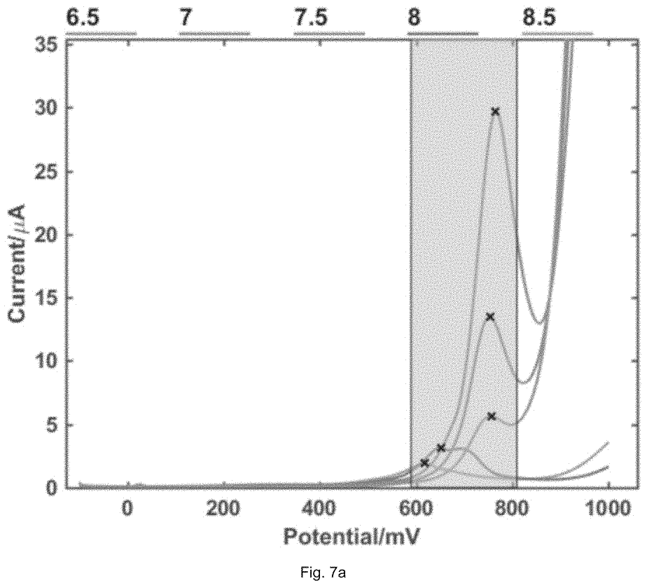

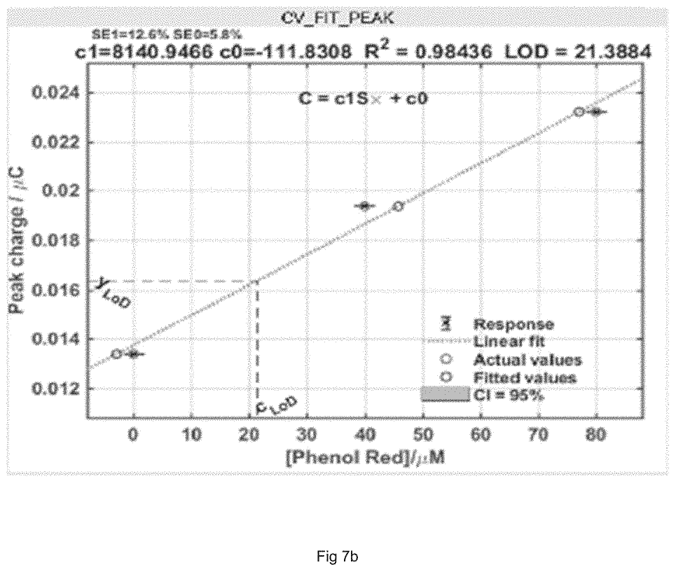

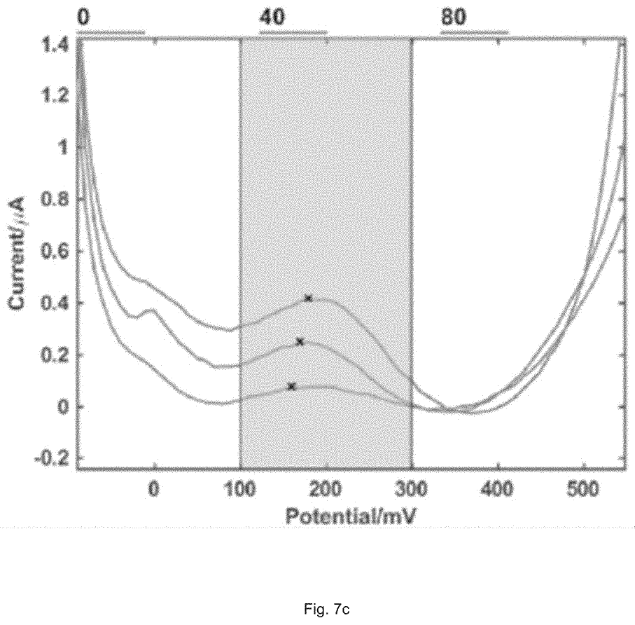

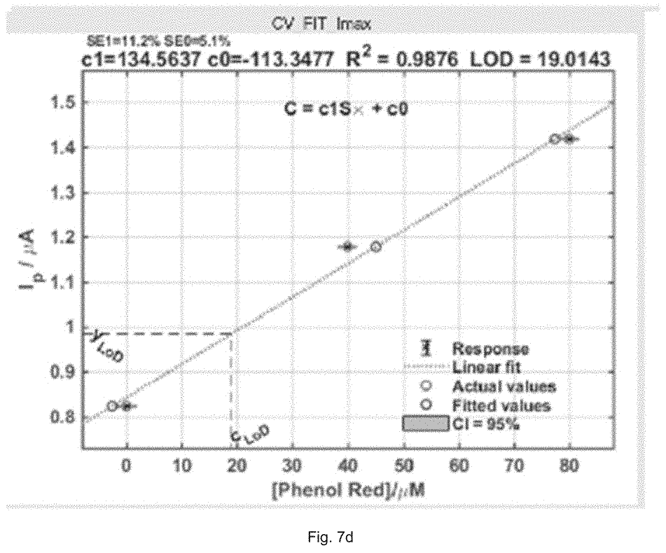

The results of performing squarewave voltammetry on phenol red in the pH range of 6.5 to 8.5, at different concentrations of phenol red, may be seen in FIG. 7. The lower two graphs of FIG. 7 show peak positions (voltage) and heights (current) versus concentrations of phenol red.

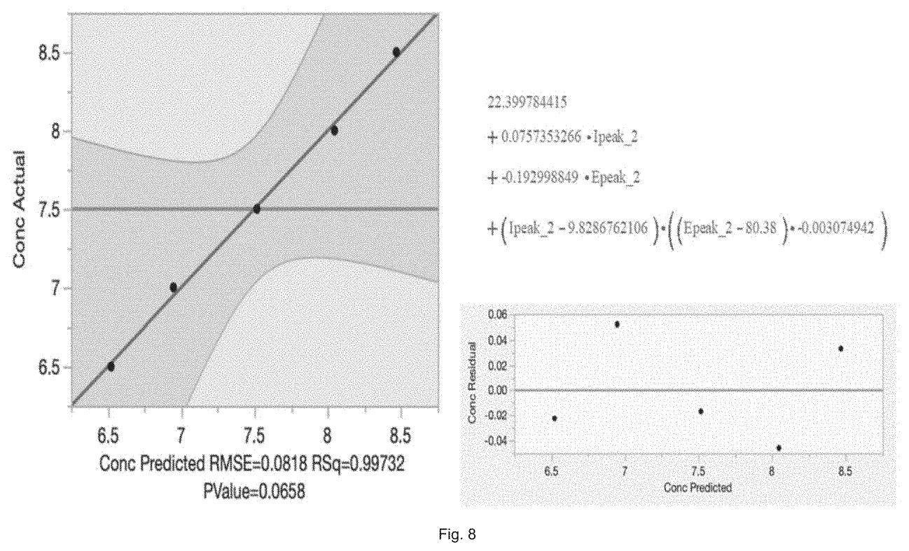

A linear regression algorithm was then applied to obtain an accurate value of pH, i.e. the output signal obtained was correlated to the pH value by application of a linear regression algorithm. This algorithm was validated with fitting the predicted value with actual value, with an error within 0.05 pH, the results of which can be seen in FIG. 8.

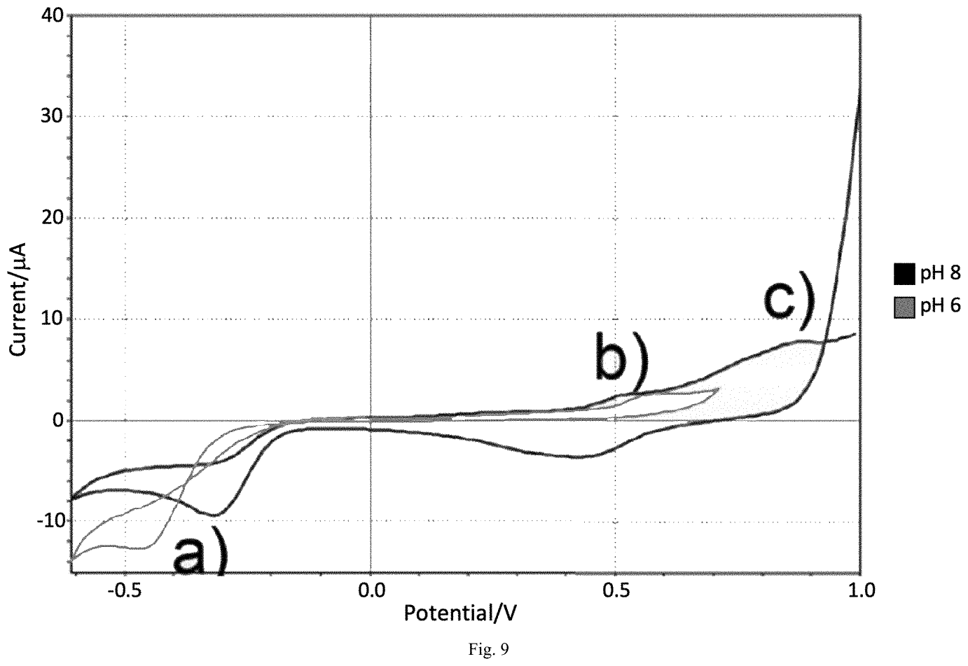

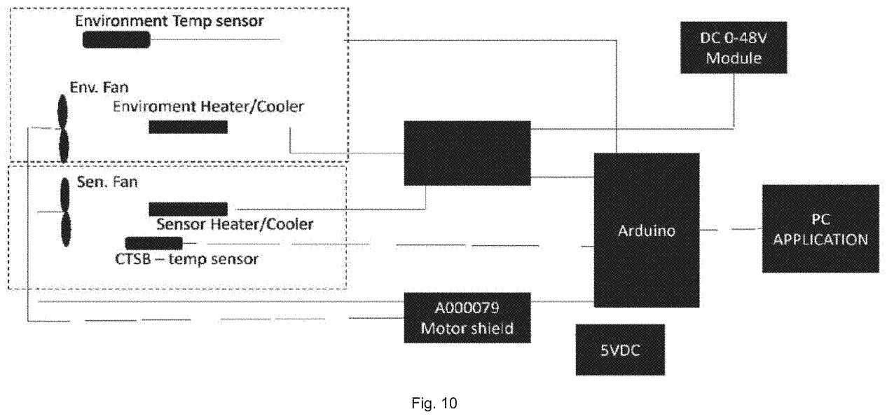

Besides the peaks in the scan between 0-800 mV, two other peaks were also observed (see FIG. 9).

Peaks a) b) and c) as highlighted in FIG. 9 are pH dependent. This study focused on peaks a) and b), as peak c) is too close to hydrolysis, which is irreversible EC peak. Peak a): From high pH to low pH: shifts to positive potential; 1 electron transfer across pH range (˜50-60 mV/pH). Peak b): From high pH to low pH: shifts to negative potential; number of electron transfer depends on pH (˜30-60 mV/pH).

The extreme accuracy of pH quantification that this method provides despite the very small sample size required has many applications, including highly accelerated monitoring and quantification of nucleic acid amplification from small samples as shown in Example 4.

### Additional Embodiments

Embodiment 1: A method of measuring the pH of a solution, wherein the method comprises the steps of:
providing a solution comprising a quinone, a quinone derivative, and/or a pH indicator;
applying the solution to a three-electrode electrochemical cell;
measuring an electrochemical response of the electrochemical cell; and
quantifying the pH of the solution as a function of the electrochemical response of the electrochemical cell.

Embodiment 2: The method according to Embodiment 1, wherein the quinone or quinone derivative is selected from the list consisting of 1,2-benzoquinone, 1,4-benzoquinone, 1,4-naphthoquinone, 9,10-anthraquinone, and derivatives and combinations thereof.

Embodiment 3: The method according to Embodiment 1 wherein the pH indicator is selected from the list consisting of malachite green oxalate, brilliant green, eosin yellowish, erythrosine B, methyl green, methyl violet, picric acid, cresol red, crystal violet, m-Cresol purple, thymol blue, p-Xylenol blue, Eosin (bluish), quinaldine red, 2,4-dinitro phenol, 4-(dimethylamino) azobenzol, bromochlorophenol blue, bromophenol blue, congo red, methyl orange, bromocresol green, 2,5-dinitrophenol, alizarin sulphonic acid, methyl red, chlorophenol red, litmus, bromocresol purple, bromophenol red, 4-nitrophenol, bromoxylenol blue, bromothymol blue, phenol red, 3-nitrophenol, neutral red, creosol red, 1-naphtholphthalein, m-cresol purple, thymol blue, p-xylenol blue, phenolphthalein, thymolphthalein, alkali blue, alizarin yellow GG, indigo carmine, epsilon blue, titan yellow, and combinations thereof.

Embodiment 4: The method according to any one of the preceding Embodiments wherein the quinone, quinone derivative, and/or pH indicator is dissolved in the solution.

Embodiment 5: The method according to any one of the preceding Embodiments, wherein the electrochemical response of the electrochemical cell that is measured is the potential of the electrochemical cell, the current of the electrochemical cell, the impedance of the electrochemical cell, or a combination of these.

Embodiment 6: The method according to any one of the preceding Embodiments, wherein the electrochemical response of the electrochemical cell that is measured comprises a combination of the potential of the electrochemical cell, the current of the electrochemical cell, the impedance of the electrochemical cell.

Embodiment 7: The method according to any one of the preceding Embodiments, wherein the potential of the electrochemical cell is measured via cyclic squarewave voltammetry, squarewave voltammetry, linear sweep voltammetry, cyclic voltammetry or open circuit potentiometry.

Embodiment 8: The method according to any one of the preceding Embodiments, wherein the concentration of the quinone, a quinone derivative, and/or a pH indicator within the solution is from about 1 μM to about 1000 μM, such as from about 1 μM to about 500 μM, for example from about 5 μM to about 250 μM, in particular from about 50 μM to about 200 μM.

Embodiment 9: The method according to any one of the preceding Embodiments, wherein the step of quantifying the pH of the solution as a function of the electrochemical response of the electrochemical cell is performed via application of a regression algorithm.

Embodiment 10: The method according to any preceding Embodiment, wherein the solution comprises a buffer.

Embodiment 11: The method according to any preceding Embodiment, wherein the solution further comprises a sample, preferably wherein the sample is selected from the list consisting of a nasal sample, a throat sample, an anal sample, a vaginal sample, an ear draining sample, a skin surface swab sample, a urine sample, a whole blood sample, a serum sample, a plasma sample and a lymph drainage sample.

Embodiment 12: The method according to any preceding Embodiment, wherein the solution further comprises primers and nucleic acid amplification reagents, preferably wherein the nucleic acid amplification reagents are LAMP reagents.

Embodiment 13: The method according to Embodiment 8, wherein the nucleic acid amplification reagents comprise a signaling substance, or are capable of releasing a signaling substance, or comprise both a signaling substance and a substance capable of release a signaling substance.

Embodiment 14: The method according to Embodiment 13, wherein the signaling substance is H+.

Embodiment 15: The method according to any one of Embodiments 12 to 14, wherein the method comprises a step of performing a nucleic acid amplification step, preferably a plurality of nucleic acid amplification steps.

Embodiment 16: The method according to any one of Embodiments 12 to 15, wherein the nucleic acid amplification step(s) is/are a loop-mediated isothermal amplification

(LAMP) step.

Embodiment 17: The method according to any one of Embodiments 12 to 16, wherein the method is for determining the presence of a target polynucleotide in the solution.

Embodiment 18: The method according to any one of Embodiments 12 to 17, wherein the step of measuring the electrochemical response of the electrochemical cell comprises measuring a change in the current and/or potential of the electrochemical cell due to a concentration change of the signaling substance.

Embodiment 19: The method according to any one of Embodiments 12 to 18, wherein the LAMP reagents comprise a reverse transcriptase.

Embodiment 20: The method according to any one of Embodiments 12 to 19, wherein the primers comprise a forward inner primer, a backward inner primer, a forward outer primer and a backward outer primer.

Embodiment 21: The method according to Embodiment 20, optionally wherein the LAMP amplification is performed with an additional pair of loop primers, such as forward loop primer and/or backward loop primer.

Embodiment 22: The method according to any of the preceding Embodiments, wherein the three-electrode electrochemical cell comprises a working electrode, a reference electrode and a counter electrode.

Embodiment 23: The method according to any one of the preceding Embodiments, wherein the working electrode comprises an ion selective membrane, located such that it excludes selected ions of the solution from contacting the working electrode. Embodiment 24: The method according to any one of the preceding Embodiments, wherein the electrodes comprise or consist of gold, silver, carbon, platinum, ruthenium dioxide, or a combination thereof.

Embodiment 25: The method according to any one of the preceding Embodiments, wherein the material of the electrodes are identical or different.

Embodiment 26: The method according to any one of the preceding Embodiments, wherein the electrodes of the three-electrode electrochemical cell are film electrodes, such as screen printed electrodes.

Embodiment 27: The method according to any one of the preceding Embodiments, wherein the electrodes of the three-electrode electrochemical cell are wire electrodes.

Embodiment 28: The method according to any one of the preceding Embodiments, wherein the three-electrode electrochemical cell comprises a substrate onto which the electrodes are located, and wherein the material of said substrate is a chemically inert material, such as alumina, ceramic, glass, or an inert plastic.

Embodiment 29: The method according to any one of the preceding Embodiments, wherein the three-electrode electrochemical cell comprises a microfluidic inlet, in fluidic connection with the electrochemical cell, for receiving the solution.

Embodiment 30: The method according to any one of the preceding Embodiments, wherein, following application of the solution, a vapour barrier is added for preventing evaporation of the solution.

Embodiment 31: The method according to Embodiment 30, wherein the vapor barrier is formed by fluidly sealing the inlet of the three-electrode electrochemical cell, or the microwell, with a physical lid, a heated lid, a mineral oil, and/or a paraffin wax.

Embodiment 32: The method according to any one of the preceding Embodiments, wherein a hydrophilic area connects the microfluidic inlet and the three-electrode electrochemical cell, such as for capillary filling of the three-electrode electrochemical cell.

Embodiment 33: The method according to any one of the preceding Embodiments, wherein the measurements are performed by using a potentiostat or similar circuit.

Embodiment 34: The method according to any of the preceding Embodiments, wherein the measurements are performed for a period of 1 minute to 90 minutes.

Embodiment 35: The method according to any of the preceding Embodiments, wherein the nucleotide amplification is performed at a constant temperature in the range of from 59 to 75° C., such as from 62 to 73° C., such as from 64 to 70° C., such as from 66 to 68° C.

Embodiment 36: Use of a three-electrode electrochemical cell for measuring the pH of a solution, said use comprising:
providing a solution comprising a quinone, a quinone derivative, a pH indicator, or combinations thereof;
applying the solution to the three-electrode electrochemical cell;
measuring an electrochemical response of the electrochemical cell; and
quantifying the pH of the solution as a function of the electrochemical response of the electrochemical cell.

Embodiment 37: Use of a three-electrode electrochemical cell for monitoring a nucleic acid amplification reaction, said use comprising:
providing a solution comprising a quinone, a quinone derivative, a pH indicator, or combinations thereof;
providing a LAMP mixture comprising: a sample comprising a target polynucleotide, at least two, such as at least four primers each flanking the target polynucleotide and LAMP reagents;
applying the solution and the LAMP mixture to the three-electrode electrochemical cell;
measuring an electrochemical response of the electrochemical cell; and
quantifying the pH of the solution as a function of the electrochemical response of the electrochemical cell.

Embodiment 38: The use according to Embodiment 37, wherein the amplification reaction is loop-mediated isothermal amplification (LAMP).

Embodiment 39: A system for measuring the pH of a solution, the system comprising:
a) a potentiostat; and
b) a measuring kit, said kit comprising:
i) a first receptacle comprising a three-electrode electrochemical cell, and a second receptacle comprising a quinone, a quinone derivative, and/or a pH indicator, or
ii) a first receptacle comprising a three-electrode electrochemical cell and a quinone, a quinone derivative, and/or a pH indicator.

Embodiment 40: The system according to Embodiment 39, wherein the quinone, quinone derivative, and/or pH indicator are dissolved in an aqueous solution in the first or in the second receptacle.

Embodiment 41: The system according to any one of the preceding Embodiments, wherein the potentiostat is configured to measure an electrochemical response of the electrochemical cell.

Embodiment 42: The system according to any one of the preceding Embodiments, wherein the potentiostat is configured to measure an electrochemical response, wherein the electrochemical response is representative of the oxidation state of the quinone, quinone derivative, and/or pH indicator in the first receptacle.

Embodiment 43: The system according to any one of the preceding Embodiments, wherein the system further comprises a heating unit, configured for heating the first and/or second receptacle, such as to a temperature within the in the range of from 59° C. to 75° C.

Embodiment 44: The system according to any one of the preceding Embodiments, wherein the first receptacle accommodates a nucleic acid amplification reaction system, such as at least four primers configured to flank a target polynucleotide sequence and LAMP reagents.

Embodiment 45: The system according to any one of the preceding Embodiments, wherein the first receptacle comprises a microfluidic inlet for receiving a solution and/or a sample.

Embodiment 46: The system according to Embodiment 44, wherein the microfluidic inlet is connected to the electrochemical cell by a hydrophilic zone, such as a porous/fibrous structure or a hydrophilic channel, configured to transport the sample by capillary action.

Embodiment 47: The system according to any one of the preceding Embodiments, wherein the first receptacle and/or the second receptacle is an Eppendorf tube, a microwell, or a culture flask.

Embodiment 48: The system according to any one of the preceding Embodiments, wherein the electrodes are as defined in any one of Embodiments 22 to 29.

1. A method of measuring the pH of a solution, wherein the method comprises the steps of:
providing a solution comprising a quinone, a quinone derivative, and/or a pH indicator, wherein the quinone, quinone derivative, and/or pH indicator is dissolved in the solution;
applying the solution to a three-electrode electrochemical cell;
measuring an electrochemical response of the electrochemical cell; and
quantifying the pH of the solution as a function of the electrochemical response of the electrochemical cell, wherein the response correlates with the electrochemical state of the quinone, quinone derivative and/or pH indicator.

2. The method according to claim 1, wherein the quinone or quinone derivative is selected from the list consisting of 1,2-benzoquinone, 1,4-benzoquinone, 1,4-naphthoquinone, 9,10-anthraquinone, and derivatives and combinations thereof.

3. The method according to claim 1, wherein the pH indicator is selected from the list consisting of malachite green oxalate, brilliant green, eosin yellowish, erythrosine B, methyl green, methyl violet, picric acid, cresol red, crystal violet, m-Cresol purple, thymol blue, p-Xylenol blue, Eosin (bluish), quinaldine red, 2,4-dinitro phenol, 4-(dimethylamino) azobenzol, bromochlorophenol blue, bromophenol blue, congo red, methyl orange, bromocresol green, 2,5-dinitrophenol, alizarin sulphonic acid, methyl red, chlorophenol red, litmus, bromocresol purple, bromophenol red, 4-nitrophenol, bromoxylenol blue, bromothymol blue, phenol red, 3-nitrophenol, neutral red, creosol red, 1-naphtholphthalein, m-cresol purple, thymol blue, p-xylenol blue, phenolphthalein, thymolphthalein, alkali blue, alizarin yellow GG, indigo carmine, epsilon blue, titan yellow, and combinations thereof.

4. The method according to claim 1, wherein the electrochemical response of the electrochemical cell that is measured is the potential of the electrochemical cell, the current of the electrochemical cell, the impedance of the electrochemical cell, or a combination of these.

5. The method according to claim 1, wherein the electrochemical response of the electrochemical cell that is measured comprises a combination of the potential of the electrochemical cell, the current of the electrochemical cell, the impedance of the electrochemical cell.

6. The method according to claim 1, wherein the potential of the electrochemical cell is measured via cyclic squarewave voltammetry, squarewave voltammetry, linear sweep voltammetry, cyclic voltammetry or open circuit potentiometry.

7. The method according to claim 1, wherein the concentration of the quinone, a quinone derivative, and/or a pH indicator within the solution is from about 1 μM to about 1000 μM.

8. The method according to claim 1, wherein the solution comprises a buffer.

9. The method according to claim 1, wherein the solution further comprises a nasal sample, a throat sample, an anal sample, a vaginal sample, an ear draining sample, a skin surface swab sample, a urine sample, a whole blood sample, a serum sample, a plasma sample or a lymph drainage sample.

10. The method according to claim 1, wherein the solution further comprises primers and nucleic acid amplification reagents, preferably wherein the nucleic acid amplification reagents are LAMP reagents.

11. The method according to claim 10, wherein the method comprises a step of performing one or more nucleic acid amplification steps.

12. The method according to claim 10, wherein the nucleic acid amplification step(s) is/are a loop-mediated isothermal amplification (LAMP) step.

13. The method according to claim 10, wherein the method is for determining the presence of a target polynucleotide in the solution.

14. The method according to claim 1, wherein the step of measuring the electrochemical response of the electrochemical cell comprises measuring a change in the current and/or potential of the electrochemical cell due to a concentration change of the signaling substance.

15. The method according to claim 1, wherein said method quantifies a change in pH of the solution.

16. The method according to claim 1, wherein the three-electrode electrochemical cell comprises a working electrode, a reference electrode and a counter electrode.

17. The method according to claim 1, wherein the electrodes comprise or consist of gold, silver, carbon, platinum, ruthenium dioxide, or a combination thereof.

18. The method according to claim 1, wherein the electrodes of the three-electrode electrochemical cell are film electrodes, screen printed electrodes or wire electrodes.

19. The method according to claim 1, wherein the measurements are performed by using a potentiostat or similar circuit.

20. The method according to claim 1, wherein the measurements are performed for a period of 1 minute to 90 minutes.
